(19) United States
(12) Patent Application Publication
(10) Pub. No.: US 2025/0104839 A1
(43) Pub. Date: Mar. 27, 2025

(54) METHODS AND SYSTEMS FOR PROVIDING A TREATMENT RESPONSE PREDICTION BASED ON A WHOLE SLIDE IMAGE

(71) Applicant: Siemens Healthineers AG, Forchheim (DE)

(72) Inventors: Svenja LIPPOK, Uttenreuth (DE); Sushil BERIWAL, Wexford, PA (US); Sven KOHLE, Erlangen (DE); Ricky Anupam SHARMA, Lightwater (GB); Marta VILALTA, Belmont, CA (US)

(73) Assignee: Siemens Healthineers AG, Forchheim (DE)

(21) Appl. No.: 18/888,482

(22) Filed: Sep. 18, 2024

(30) Foreign Application Priority Data
Sep. 22, 2023 (DE) 10 2023 209 304.9

(51) Int. Cl. G16H 20/40; G16H 10/60; G16H 30/00

(52) U.S. Cl. CPC G16H 20/40; G16H 10/60; G16H 30/00

(57) ABSTRACT

A computer-implemented method for providing a treatment response prediction for a patient suffering from a cancerous disease, comprises: obtaining a whole slide image of the patient showing a tissue sample relating to the cancerous disease; providing a prediction function configured to derive a treatment response prediction for one or more treatment options from whole slide images; and applying the prediction function to the whole slide image to provide the treatment response prediction.

### CROSS-REFERENCE TO RELATED APPLICATION(S)

The present application claims priority under 35 U.S.C. § 119 to German Patent Application No. 10 2023 209 304.9, filed Sep. 22, 2023, the entire contents of which is incorporated herein by reference.

### FIELD

Various examples of the disclosure relate to providing a treatment response prediction based on a whole slide image. Specifically, various examples of the disclosure relate to methods and systems configured to process whole slide images in order to derive a treatment response prediction. In other words, examples of this disclosure relate to an automated processing of image data in order to provide a clinically actionable result/diagnosis.

### BACKGROUND

Radiation therapy is commonly applied to the cancerous tumor because of its ability to control cell growth. Ionizing radiation works by directly and indirectly damaging the DNA of cancer cells leading to cellular death. To spare normal tissues such as skin and organs which radiation must pass through to treat the tumor, shaped radiation beams are aimed from several angles of exposure to intersect at the tumor, providing a much larger absorbed dose there than in the surrounding healthy tissue.

Radiation therapy is synergistic with chemotherapy, immunotherapy, and other cancer therapies, and has been used before, during, and after other treatments in susceptible cancers.

The question whether or not a radiotherapy treatment is to be used (i.e., whether a tumor is susceptible to radiotherapy) and the orchestration of a radiotherapy treatment plan with other treatment options is a difficult question which requires a lot of experience at the side of the radiation oncologist and critically affects the patient's wellbeing.

Moreover, in the clinical routine, physicists have to base the decision for a particular treatment on a plethora of additional factors. In addition, physicists not only consider one treatment option but have to weigh a plurality of options. This has the consequence that physicists have to manage a huge amount of data and information. What is more, one issue with current decision assistance systems is that it is often not evident for a user how reliable a certain outcome of such an automated processing is.

### SUMMARY

Accordingly, it is an object of one or more embodiments of the disclosure to provide methods and systems for automatically predicting a treatment response of a cancer patient for one or more treatment options coming into question. In particular, it is an object of one or more embodiments of the disclosure to enable a reliable treatment response prediction by efficiently using the available data for the patient.

At least this object is solved by a method for providing a treatment response prediction a corresponding system, corresponding non-transitory computer-program products, and non-transitory computer-readable storage media according to the claims. Alternative and/or preferred embodiments are object of the dependent claims.

In the following, a technical solution according to embodiments of the present invention is described with respect to the claimed apparatuses as well as with respect to the claimed methods. Features, advantages, or alternative embodiments described herein can likewise be assigned to other claimed objects and vice versa. In other words, claims addressing the inventive method can be improved by features described or claimed with respect to the apparatuses. In this case, e.g., functional features of the method are embodied by objective units or elements of the apparatus.

The technical solution will be described both with regard to methods and systems for providing information and also with regard to methods and systems for providing prediction functions. Features and alternate forms of embodiments of data structures and/or functions for methods and systems for providing predictions can be transferred to analogous data structures and/or functions for methods and systems for providing prediction functions. Analogous data structures can, in particular, be identified by using the prefix “training”. Furthermore, the prediction functions used in methods and system for providing information can, in particular, have been adjusted and/or trained and/or provided by methods and systems for adjustment of prediction functions.

According to an aspect, an embodiment of a computer-implemented method for providing a treatment response prediction for a patient suffering from a cancerous disease is provided. The method comprises a plurality of steps. One step is directed to obtain a whole slide image of the patient showing a tissue sample relating to the cancerous disease. Another step is directed to provide a prediction function configured to derive a treatment response prediction for one or more treatment options from whole slide images. Another step is directed to apply the prediction function to the whole slide image so as to generate the treatment response prediction. Another step is directed to provide the treatment response prediction.

The cancerous disease may relate to a neoplasm (also denoted as “tumor”), in particular, a benign neoplasm, an in-situ neoplasm, a malignant neoplasm and/or a neoplasm of uncertain/unknown behavior associated to the patient's body.

In particular, the cancerous disease may comprise a neoplasm of inner organs of the patient, such as a lung tumor, prostate cancer, lymph node cancer, and so forth.

Whole-slide images to be processed by the prediction function may be two-dimensional digital images having a plurality of pixels. Whole slide images may have a size of at least 4.000×4.000 pixels, or at least 10.000×10.000 pixels, or at least 1E6×1E6 pixels.

A whole-slide image may image a tissue slice or slide of a patient. The preparation of the tissue slices from the tissue samples can comprise the preparation of a section from the tissue sample (for example with a punch tool), with the section being cut into micrometer-thick slices, the tissue slices. Another word for section is block or punch biopsy. Under microscopic observation, a tissue slice can show the fine tissue structure of the tissue sample and, in particular, the cell structure or the cells contained in the tissue sample. When observed on a greater length scale, a whole-slide image can show an overview of the tissue structure and tissue density. The tissue may have been taken from a tumor the patient is suffering from. In particular, the tissue may show a manifestation of a cancerous disease of the patient, such cells of a tumor.

The preparation of a tissue slice further may comprise the staining of the tissue slice with a histopathological staining. The staining in this case can serve to highlight different structures in the tissue slice, such as, e.g., cell walls or cell nuclei, or to test a medical indication, such as, e.g., a cell proliferation level. Different histopathological stains are used for different purposes in such cases.

To create the whole-slide image, the stained tissue slices are digitized or scanned. To this end, the tissue slices are scanned with a suitable digitizing station, such as, for example, a whole-slide scanner, which preferably scans the entire tissue slice mounted on an object carrier and converts it into a pixel image. In order to preserve the color effect from the histopathological staining, the pixel images are preferably color pixel images. Since in the prediction both the overall impression of the tissue and also the finely resolved cell structure may be of significance, the whole slide images typically have a very high pixel resolution. The data size of an individual image can typically amount to several gigabytes.

A treatment response prediction may be conceived as a readout of how promising a certain treatment option or therapy is for the patient given the whole slide image. It may comprise a number proportional to an anticipated success rate of at least one treatment option. According to some examples, the number may comprise a percentage. The percentage may signify a match, suitability, or success rate of the underlying treatment option in view of the whole slide image.

The treatment response prediction may comprise an indication for one or more treatment options. In particular, the treatment response prediction may comprise a readout for each one of a plurality of treatment options.

A treatment option may be a therapy or part of a therapy coming into question for treating the cancerous disease of the patient. The therapies may be pre-defined. Different treatment options may differ in providing different kinds of treatments or in providing the same kind of treatment but with different parameters such as different dosage regimes of a particular medication.

According to some examples, one or more of the treatment options may relate to a treatment administrated to the patient before a tissue sample forming the basis for the whole slide image was taken.

The prediction function may generally be conceived as an image processing function configured to process image data comprised in whole slide images. The prediction function may be configured to classify the whole slide image according to one or more of the treatment options.

By basing the treatment prediction on whole slide images, a resource can be leveraged which is available for the majority of cancer cases. Moreover, depicting a cancerous growth on a cell level, whole slide images give insights in the prospective behavior of the tumor cell—and, therewith, the tumor, vis-à-vis different treatment options. Further, the automated processing using a prediction function is capable of providing an objective readout and spares the user from the task of manually selecting and evaluating different options.

According to some examples, the prediction function is configured to extract one or more features from whole slide images and generate the treatment response prediction based on the extracted features.

The extracted features may give cues about how the underlying cancerous disease would respond to one or more of the treatment options. The feature categories (or kinds of features) may be predetermined and, in particular, learned. The one or more features may be integrated in a so-called feature vector, or, in this case histopathological feature vector. The one or more features may also be designated as digital biomarker characterizing the whole slide images according to the one or more treatment options.

The one or more features may describe or may be based on the morphology of the tissue and, in particular, the cancerous cells depicted in the whole slide image. In other words, one or more of the features may comprise morphological information, e.g., shape, size, color, composition of tumor cells, or tumor infiltration, specific markers, e.g., used for information on a proliferation status or immune checkpoint inhibition, information on a response to an existing therapy, e.g., a percentage of necrotic tissue (e.g., no necrotic tissue means no response and indicates that a certain treatment is not efficient). One or more of the features may be tangible to a human user, i.e., may be directly interpretable for a human user. Further, one or more of the features may be abstract with no apparent meaning for a human user. According to some examples, the one or more features may be semantic features.

According to some examples, generating the treatment response prediction may comprise comparing the one or more features to one or more of the treatment options and/or selecting at least one treatment option from a plurality of treatment options based on the one or more features. Comparing may comprise comparing the one or more features to corresponding characteristics associated to the respective treatment options. In other words, the one or more features may be classified according to the one or more treatment options to generate the treatment response prediction.

According to some examples, the prediction function may be configured to map the features to higher-level or meta features, which characterize the whole slide image according to one or more categories relevant for selecting a treatment option, like susceptibility to a treatment option, recurrence probability with the treatment option, survival rate or other more abstract meta features. The process of mapping may also be called classifying. Based on these meta features, the treatment response prediction may be provided. According to some examples, this may involve selecting one or more treatment options based on the meta features. According to some examples, one or more of the meta features may be provided as part of the treatment response prediction.

According to some examples, the method further comprises: respectively defining a plurality of tiles in the whole slide image, wherein the prediction function is configured to extract one or more features for each one of at least part of the tiles, and generate the treatment response prediction based on the features extracted, in particular, by calculating average features from the features extracted.

The tiles may be conceived as subareas or patches of the respective whole slide image. Tiles also comprise image data in the form of pixels. Tiles are typically much smaller than the underlying whole slide images. Tiles may be defined by laying a regular grid over the underlying whole slide image. Each tile may have the same size. With the tile-wise processing, the large whole-slide images may be portioned in smaller parts which are easier to process from a computational perspective.

According to some examples, the prediction function may be a trained function.

In general, a trained functions mimics cognitive functions that humans associate with other human minds. In particular, by training based on training data, the trained functions are able to adapt to new circumstances and to detect and extrapolate patterns. Other terms for trained function, may be machine-learned or machine learning function (abbreviated by ML function), trained machine learning model, trained mapping specification, mapping specification with trained parameters, function with trained parameters, algorithm based on artificial intelligence, or machine learned algorithm (abbreviated by ML algorithm).

In general, parameters of a trained function can be adapted by way of training. In particular, supervised training, semi-supervised training, unsupervised training, reinforcement learning and/or active learning can be used. Furthermore, representation learning (an alternative term is “feature learning”) can be used. In particular, the parameters of the trained functions can be adapted iteratively by several steps of training.

In particular, a trained function can comprise a neural network, a support vector machine, a decision tree and/or a Bayesian network, and/or the trained function can be based on k-means clustering, Qlearning, genetic algorithms and/or association rules. In particular, a neural network can be a deep neural network, a convolutional neural network or a convolutional deep neural network. Furthermore, a neural network can be an adversarial network, a deep adversarial network and/or a generative adversarial network.

As a general rule, the neural networks include multiple layers. The input to a first layer is the input image (in this case a whole slide image). Each layer can apply one or more mathematical operations on the input values, e.g., convolutions, nonlinear excitations, pooling operations, to give just a few examples. The input to a layer can be formed by the output of a preceding layer (feed-forward). Feedback of values or skip-connection skipping layers are possible.

A neural network for digital pathology, i.e., as may be comprised in the prediction function, may be configured to infer at least one feature from whole slide images. In particular, it would be possible to employ a convolutional neural network as trained function in the prediction function. For instance, ResNet-18 may be used, see Ayyachamy, Swarnambiga, et al. “Medical image retrieval using Resnet-18.” Medical Imaging 2019: Imaging Informatics for Healthcare, Research, and Applications. Vol. 10954. International Society for Optics and Photonics, 2019. Furthermore, a VGG-16 or VGG-19 CNN could be used, see: Mateen, Muhammad, et al. “Fundus image classification using VGG-19 architecture with PCA and SVD.” Symmetry 11.1 (2019): 1; or Kaur, Taranjit, and Tapan Kumar Gandhi. “Automated brain image classification based on VGG-16 and transfer learning.” 2019 International Conference on Information Technology (ICIT). IEEE, 2019.

According to some examples the prediction function may comprise a convolutional neural network. A convolutional neural network is a neural network that uses a convolution operation instead general matrix multiplication in at least one of its layers (so-called “convolutional layer”). In particular, a convolutional layer performs a dot product of one or more convolution kernels with the convolutional layer's input data/image, wherein the entries of the one or more convolution kernel are the parameters or weights that are adapted by training. In particular, one can use the Frobenius inner product and the ReLU activation function. A convolutional neural network can comprise additional layers, e.g., pooling layers, fully connected layers, and normalization layers.

According to some examples, the prediction function may comprise a ResNet architecture. ResNet stands for Residual Neural Network. ResNet is a deep learning model in which the weight layers learn residual functions with reference to the layer inputs. A Residual Network is a network with skip connections that perform identity mappings, merged with the layer outputs by addition. This enables ResNets with tens or hundreds of layers to train easily and approach better accuracy.

ResNets may comprise a plurality of convolutional layers for processing. According to some examples, the prediction function may comprise a so-called ResNet-50. ResNet-50 is a 50-layer convolutional neural network comprising 48 convolutional layers, one Max pooling layer, and one average pooling layer.

Training data for training a prediction function may comprise data sets of a plurality of patients (>1000) suffering from a particular cancerous disease. The data set of each patient may respectively comprise at least one whole slide image as well as an information about the treatment option followed and the respective outcome. The outcome may be a long-term survival rate, a prognosis, a disease progression and so forth which may be leveraged for the treatment response prediction. The outcome and therewith a ground truth for the training may be annotated by human experts.

The patient data may be clustered according to the treatment option respectively followed. For each treatment option, the prediction function may then learn to predict the treatment response based on the respective whole slide image and, optionally, any additional data.

According to some examples, providing the treatment response prediction may comprise outputting the treatment response prediction to a user via a user interface.

According to an aspect, the one or more treatment options comprise at least one of: a radiotherapy treatment, an immunotherapy treatment, a chemotherapy treatment, and/or a treatment by surgical intervention.

Each treatment option may specify parameters of the corresponding treatment. In the case of a radiotherapy treatment this may comprise radiotherapy settings, such as dose, beam geometry, dose rate, etc., which may be input in a radiotherapy treatment modality. In the case of chemotherapy treatment, this may comprise a type and dosage of a medication to be administrated to the patient. In the case of immunotherapy, this may comprise the kind of immunomodulator to be used.

According to some examples, the prediction function is configured to derive a treatment response prediction for a plurality of predefined treatment options.

This has the advantage that the prediction function can be more specifically adapted which may yield better prediction results.

The predefined treatment options may relate to standard treatment options coming into question for a particular cancerous disease. The treatment options may be predefined based on a medical guideline, prior treatments of other patients suffering from the same cancerous disease, insurance policies, and the like.

According to some examples, the prediction function may be configured to select, from the plurality of predefined treatment options, at least one treatment option based on the whole slide image (or any supplementary information) and provide the at least one selected treatment option as treatment response prediction.

According to some examples, a treatment option may comprise a combination of the aforementioned options, e.g., a combined treatment comprising a radiotherapy treatment and a chemotherapy treatment.

According to some examples, a treatment option may comprise a treatment plan involving different treatment steps over the course of a predetermined period of time. The treatment steps may relate to a radiotherapy treatment, an immunotherapy treatment, a chemotherapy treatment, a treatment by surgical intervention, and/or any combination thereof.

According to other examples, a plurality of treatment options may be combined based on the treatment response prediction to form a treatment plan and the treatment plan may be provided/outputted to a user.

According to some examples, treatment options may be combined in a treatment plan based treatment response prediction (e.g., comprising an estimated success rate per treatment option) and clinical compatibility. Combining different treatment options makes it possible to select the most promising therapy forms for the patients, which may be advantageous if, based on the treatment response prediction, there is no clear favorite.

According to some examples, the treatment response prediction comprises: a predicted susceptibility of the cancerous disease to the one or more treatment options, a probability for a reoccurrence of the disease based on the one or more treatment options, and/or a predicted survival rate of the patient with the one or more treatment options.

The susceptibility and/or the probability for recurrence may be provided as a number, such as a percentage, or in the form of a more abstract category, such as high, medium, or low.

The survival rate may be provided in the form of a survival probability of the patient within a given timeframe which may be measured in years.

According to some examples, calculating the predicted survival rate may be based on a comparison of the patient (e.g., based on the whole slide image, features derived therefrom, or any supplementary information) to a cohort of patients suffering from the same cancerous disease who already underwent treatment.

The inventors have recognized that the morphology of the cells shown in whole slide images can generally provide an indication of how cells would react to a certain kind of therapy, for instance, to ionizing radiation involved in radiation therapy. Predicting the susceptibility (another word may be vulnerability) of the tumor to a treatment option based on whole slide images thus may indicate how promising a certain therapy is and serves as a valuable measure for the treatment response prediction.

Regarding the probability for a recurrence, the inventors have recognized that this measure depends on both the morphology of the cancerous disease as visible in the whole slide images and the treatment option. This measure may provide additional information for the user to decide about the best treatment option(s) for the patient.

The survival probability has the advantage that a probability for a reoccurrence of the disease as well as side effects of the treatment options may be factored in.

According to an aspect, the method further comprises providing a certainty calculation module configured to output a certainty measure for a corresponding treatment response prediction, the certainty measure measuring the confidence of the corresponding treatment response prediction, applying the certainty calculation module to obtain a certainty measure for the treatment response prediction, and providing the certainty measure.

A certainty measure (or confidence measure, or confidence level, or certainty score, or trust level) may indicate how confident the prediction function is in predicting the treatment response for the underlying whole slide image.

A certainty measure, according to some examples, may comprise a numerical value or a collection of numerical values (e.g., a vector) that indicate, according to a model or algorithm, the degree of certainty or uncertainty a certain treatment response prediction or part of a treatment response prediction is afflicted with and/or, generally, the quality of a certain treatment response prediction. According to some examples, obtaining the certainty measures may comprise assigning a value/certainty measure to each element of the treatment response prediction.

According to some examples, a certainty measure may be provided for the treatment response prediction as whole. According to other examples, a certainty measure may be provided per element of the treatment response prediction, in particular, per treatment option comprised in the treatment response prediction.

The certainty calculation module may be applied to the whole slide image and/or the treatment response prediction and/or any intermediate processing results of the prediction function.

By introducing certainty measures, the quality of the treatment response prediction can be assessed and quantified. This allows for a more informed decision of the user on the available treatment options.

According to some examples, the certainty calculation module may be a trained function. It may have been trained to derive at least one certainty measure for a treatment response prediction based on the whole slide image and/or the behavior of the prediction function.

According to an aspect, the certainty calculation module may be integrated in the prediction function.

Specifically, the certainty measure can be based on the output layer of the prediction function. For instance, a possible confidence measure may be the output of a logit function, which can be implemented in the final layer of an artificial neural network of the respective prediction function. A more sophisticated confidence measure can be computed on the basis of a probabilistic interpretation of the output of the logit function.

Alternatively, certainty measures may be obtained using a separate module. Specifically, suchlike module may comprise a softmax unit, which is configured to determine at least one certainty measure based on a softmax action selection, and/or comprises a Bayesian unit, which is configured to determine at least one certainty measure based on Bayesian inference.

In a softmax action selection a statistical approach is taken. One can, e.g., assume that the logits output by the logit function follow a Boltzmann distribution and then associate the confidence measure to a likelihood found using logistic regression. Another possibility is to use Bayesian inference. There are different Bayesian-based approaches available, which are purported to yield very strong confidence levels associated with the predictions of artificial neural networks.

According to some examples, the certainty calculation module comprises a Bayesian model.

Such a Bayesian model is based on an artificial Bayesian network. A Bayesian network is a probabilistic graphical model that represents a set of variables and their conditional dependencies via a directed acyclic graph with the variables as vertices or nodes and the dependencies as edges. Each node of the network is assigned to a conditional probability distribution of the random variable which it represents, given the random variables at the parent node. The distribution can be arbitrary. However, discrete or normal distributions are often used. Parents of a vertex are those vertices with an edge leading to the vertex. A Bayesian network is used to represent the common probability distribution of all variables involved as compactly as possible using conditional dependencies. The conditional (in) dependency of subsets of the variables is combined with a priori knowledge.

The Bayesian model is trained to identify uncertain prediction results and, therewith, a corresponding uncertain data basis. Uncertain data are, for instance, whole slide images which the prediction function is not able to classify according to one or more treatment options with sufficient reliability. Uncertainty can be determined by extracting feature data at different layers of the Bayesian model and determining a statistical distribution based on these data. Then, uncertainty can be calculated as a standard deviation of the mentioned statistical distribution.

Bayesian models enable to model a causal relationship among random variables using conditional probabilities. Thus, they are useful to understand how an individual inaccuracy or failure propagates through the layers of the trained function and how significantly it affects the outcome. As such, Bayesian models are capable of reliably predicting the certainty or confidence of output provided by complex systems like the prediction function.

According to some examples, the certainty calculation module comprises:
a feature pyramid pooling algorithm,
a Monte Carlo Dropout algorithm,
a Monte Carlo Depth algorithm, and/or
a Deep Ensemble algorithm.

The method of pyramid pooling is described in Hanchao Li, Pengfei Xiong, Jie An, and Lingxue Wang, “Pyramid attention network for semantic segmentation”, arXiv-preprint arXiv:1805.10180. Feature pyramid pooling is used to perform spatial pyramid attention structure on high level output and combining global pooling to learn a better feature representation and a global attention upsample module on each decoder layer to provide global context as a guidance of low-level features to select category localization details. The feature pyramid pooling algorithm is used with feature maps as input data and output data. The mentioned input and output data, appropriately prepared as labelled data, can be also used as training data for training the feature pyramid pooling algorithm.

The Monte-Carlo dropout and/or Monte Carlo Depth algorithm and/or Deep Ensemble learning approach is used for deter-mining uncertain data. A Monte-Carlo dropout method includes an extraction of feature data in different layers of an artificial neural network. The extracted feature data are used to determine a statistical distribution of the features also called sample variance. Based on the statistical distribution, an uncertainty can be calculated as standard deviation.

The Monte Carlo Dropout algorithm receives feature maps of different stages of the uncertainty model as input data and generates feature maps as output data. The mentioned input and output data, appropriately prepared as labelled data, can be also used as training data for training the Monte Carlo Dropout algorithm.

The Monte Carlo Depth algorithm also receives feature maps of images as input data and output data. The mentioned input and output data, appropriately prepared as labelled data, can be also used as training data for training the Monte Carlo Depth algorithm.

Deep Ensemble works with a plurality, for example 15, base-learners. These base-learners include parallel working AI-based models, preferably artificial neuronal networks. Then Bayesian inference is carried out on these base-learners. Based on the statistics of different results of different base-learners, sample variance is used as an uncertainty metric. The Deep Ensemble algorithm is performed using a set of input data including different subsets of image data or feature maps for feeding the so-called base-learners. Further, also the output data include different subsets of image data or feature maps, or alternatively a map indicating sample variance of the input data. The mentioned input and output data, appropriately prepared as labelled data, can also be used as training data for training the Deep Ensemble algorithm.

The aforementioned examples for certainty modules enable to reliably attribute confidence values to the treatment response prediction. Thereby, different examples may bring about different advantages, for instance Monte Carlo Dropout or Monte Carlo Depth are more interwoven with the trained function. Therefore, they may require less training. Deep Ensemble is more independent requiring fewer modifications to the trained functions.

According to some examples, the certainty calculation module comprises a novelty detection algorithm configured to detect input and/or output parameters of the prediction function which are underrepresented in the training of the prediction function, and the step of obtaining the certainty measures comprises computing the certainty measure based on the detection results of the novelty detection algorithm.

In other words, the novelty detection algorithm may be configured to detect, in particular, input data, in particular, input image data, the prediction function is not used to in the sense of that it has not seen that kind of input data during training to a significant extent or at all.

According to some examples, the novelty detection may be configured to detect an incongruity in the behaviour of the prediction function.

According to some examples, the novelty detection algorithm may again be based on a Bayesian model. Specifically, e.g., through unsupervised learning, an endogenous and unsupervised Bayesian model may be trained that represents interrelations between image data and the processing of the image data by the prediction function. This Bayesian model may then be used to detect an incongruity of a behaviour of the prediction function.

According to some examples, the novelty-detection algorithm comprises an autoencoder neural network algorithm comprising an encoder branch configured to encode an input data and a decoder branch configured to decode an encoded representation of the input data output by the encoder branch, wherein an anomaly is detected based on a reconstruction error between the input data and an output data output by the decoder branch.

With the usage of a novelty detection algorithm, circumstances can be automatically identified which are less familiar or not familiar for the prediction function. Under such circumstances, it is likely that the prediction function will not be able to deliver confident results. Thus, identifying these circumstances leads to reliable certainty measures.

According to an aspect, the step of providing comprises outputting the confidence measure together with the treatment response prediction to a user via a user interface.

With the provision of the confidence measure the user is provided with direct information how “trustworthy” the results of the automated processing are. Accordingly, the user may be provided with additional information for deciding about the optimal treatment for the patient.

According to an aspect, the method further comprises determining, based on the confidence measure, whether or not the treatment response prediction is conclusive, and if the treatment response prediction is not conclusive: determine a piece of information relating to the patient which is suited for rendering the treatment response prediction conclusive.

The piece of information may be information not comprised in the whole slide image. In particular, the piece of information may relate to non-whole-slide image data. In particular, the piece of information may relate to supplementary information about the patient. The piece of information may relate to information from the electronic health record of the patient which could be potentially helpful for increasing the confidence of the treatment response prediction. The piece of information may relate to hypothetical information which may or may not be already available.

According to some examples the piece of information may comprise at least one of: a medical report of the patient, a further whole slide image different from the whole slide image, wherein, optionally, the further whole slide image may be stained with a different histopathological stain as the whole slide image, radiological image data, demographic information of the patient such as age, gender, co-morbidities, risk-factors, etc., lab data, and/or omics data.

By determining the piece of information, it is not only determined why a treatment response prediction is not conclusive and but also what could theoretically make it more conclusive. This is helpful for the user in order to decide about the treatment options and to decide about the next steps.

According to other examples, the piece of information may relate to an anamnestic finding for the patient, which the user may know. Accordingly, the method may comprise: formulating an anamnestic question directed to the piece of information, providing the anamnestic question to a user via a user interface, (optionally) receiving an answer from the user via the user interface, (optionally) modifying the treatment response prediction (e.g., by inputting the answer in the prediction function), (optionally) modifying the certainty measure (e.g., by applying the certainty calculation module), and, (optionally) providing the modified treatment response prediction and/or certainty measure (e.g., to the user via the user interface).

According to an aspect, the method further comprises providing an indication of the piece of information to a user via the user interface. This not only renders the processing more transparent but also provides a further actionable result with which the user may initiate the necessary steps for obtaining the piece of information.

According to some examples, determining whether or not the treatment response prediction is conclusive may comprise comparing the confidence measure to a predetermined threshold and determining whether or not the treatment response prediction is conclusive based on the comparison. Specifically, the treatment response prediction may be determined as not conclusive if the confidence measure, in the step of comparing, is found to be below the predetermined threshold.

According to some examples, the uncertainty calculation module may be configured to output a piece of information and the piece of information is determined based on applying the uncertainty calculation module.

Specifically, as explained above, the uncertainty calculation module may be configured to determine an uncertain data basis in the processing of the prediction function. In turn, this may provide information on data elements which would make the prediction more certain. For instance, if the prediction function has been trained to base the prediction on a certain data element which is not available for the present case, the uncertainty calculation module may determine that the data element is not there and what consequences this has for the certainty of the prediction. On that basis, it may then be determined if the missing data element needs to be flagged.

According to some examples, the prediction function is configured to provide the treatment response prediction in the form of a set of probabilities, wherein each probability of the set of probabilities reflects the probability that the cancerous disease is responsive to one of a plurality of predefined treatment options, and the piece of information is selected based on the set of probabilities, and, in particular based on one or more statistical properties of the probability distribution of the set of probabilities.

The statistical properties may comprise at least one of the entropy, variance, skewness, kurtosis and so forth of the probability distribution.

According to some examples, the piece of information is selected based on a predictive impact of the piece of information on the probabilities. In particular, this may involve determining a hypothetical change of the one or more statistical properties if the piece of information would be available.

According to some examples, the predictive impact is defined as the predictive information gain associated with each piece of information, wherein the predictive information gain for a piece of information is, in particular, given by:
MAX {P(Res1) [H(PD|WSI)−H(PD|WSI, PI=Res1)],
P(Res2) [H(PD| WSI)−H(PD|TPD, PI=Res2)], . . . ,
P(Resn) [H(PD|WSI)−H(PD|WSI, PI=Resn)}
with:
   H(PD|WSI) being the initial entropy H of the probability distribution PD of the calculated probabilities for treatment options based on the whole slide image;
   H(PD|WSI, PI=Res1) being the entropy H of the probability distribution PD assuming that the piece of information PI indicates a first result Res1;
   H(PD| WSI, PI=Res2) being the entropy H of the probability distribution PD assuming that the piece of information PI indicates a second result Res2;
   H(PD|WSI, PI=Res1) being the entropy H of the probability distribution PD assuming that the piece of information PI indicates a n-th result Resn;
question Q; and
   P(Res1), P(Res2), . . . , P(Resn) respectively being the probability that the piece of information indicates first to n-th results.

According to the above embodiment, the entropy is used as statistical property of the distribution of probability and the MAX-function is used as the metric for weighting possible results indicated by the piece of information. With that, a reliable and reproducible way of identifying relevant pieces of information may be defined.

According to an aspect, the method further comprises accessing a healthcare information system comprising an electronic medical record of the patient, retrieving the piece of information from the healthcare information system, processing the piece of information so as to provide an updated treatment response prediction and, optionally, an updated confidence measure, and providing the updated treatment response prediction and, optionally, the updated confidence measure.

Processing the piece of information may comprise inputting the piece of information into the prediction function and/or the certainty calculation module.

By automatically fetching and processing the piece of information, the user can be further relieved as he is provided with an improved treatment response prediction without the need to interfere.

According to some examples, the piece of information comprises radiology image data depicting a manifestation of the cancerous disease in the body of the patient.

According to some examples, the step of processing the piece of information comprises: providing an image analysis module configured to extract a radiological observable from radiology image data, applying the image analysis module on the radiology image data so as to obtain the radiological observable, and generating the updated treatment response prediction and, optionally, the updated confidence measure, additionally based on the radiological observable. Thereby, the radiological observable may be of the form as herein described.

According to an aspect, the method further comprises accessing a healthcare information system comprising healthcare data (an electronic medical record) of the patient, determining if the piece of information is available in the healthcare information system, if the piece of information is not available: generating a corresponding notification to the user.

With the notification, the user can be automatically pointed to information which would be beneficial for arriving at more certain treatment response prediction. This enables the user to initiate the necessary steps in order to obtain the piece of information.

According to an aspect, the piece of information relates to a medical examination of the patient, and the method further comprises generating an instruction for performing the medical examination, and providing the instruction.

The instruction may comprise human-readable and/or machine readable instructions. For instance, the instructions may comprise steps and/or parameters for performing the medical examination.

A medical examination may comprise an examination at the patient's body for obtaining one or more measurements. The measurements may be used as the piece of information. The examination may comprise a radiological examination, a laboratory examination of a patient sample, a genomics examination of a patient sample, a (further) histopathological examination, or a general examination. The medical examination may be performed with a corresponding modality configured for performing the medical examination, such as a radiological imaging modality, a laboratory test modality, a genomic test modality and so forth. The modality may be connected to the healthcare information system.

According to some examples, the instructions comprise human-readable instructions and the instructions are provided to a user via a user interface.

According to some examples, the instructions comprise machine-readable instructions configured to control a modality configured to perform the medical examination to perform the medical examination and the method further comprises: inputting/forwarding the instructions in/to the modality (so as control the modality to perform the medical examination), and/or providing the instruction to a user via a user interface (for review and further usage).

According to some examples, the medical examination may comprise generating a further whole slide image different than the whole slide image.

The further whole slide image may differ from the whole slide image in that it has been stained with a histopathological stain different than the one used for the whole slide image.

By basing the prediction on differently stained whole slide images, complementary image information is provided which can improve the prediction. As each histopathological stain highlights specific structures in a tissue slice, the histopathological stain of the further whole slide image can be used to specifically highlight structures which are helpful for ruling out or substantiating certain treatment options. In particular, the histopathological stain of the further whole slide image may be a stain which is less common in the clinical routine as compared to the histopathological stain of the whole slide image and which may be obtained only on a request basis if the certainty measure is too low.

According to some examples, the histopathological stain of the whole slide image is a H&E stain, and/or the histopathological stain of the further whole slide image is an immunohistochemistry stain, in particular, comprising keratin targeting biomarkers.

In this regard, H&E stands for hematoxylin and eosin. Hematoxylin stains cell nuclei, and Eosin stains the extracellular matrix and cytoplasm. H&E is the most widely used stain in digital pathology—making also the initial prediction widely applicable.

Immunohistochemistry stains, or short IHC stains, involve the process of selectively identifying antigens (proteins) in cells of a tissue section by exploiting the principle of antibodies binding specifically to antigens in biological tissue. With that, structures can be highlighted which are not accessible with other stains such as H&E. Accordingly, an additional readout may be provided for further detailing the prediction of treatment responses.

In particular, the IHC stain may comprise biomarkers (e.g., in the form of antibodies) configured to target (cytoskeletal) keratins. Keratins form part of the cell cytoskeleton and define the mechanical properties of cells. As such, the abundance of keratins makes up a good tumor marker and may characterize the “aggressivity” of tumor cells as keratin expression levels are often altered in tumor cells. Specifically, the IHC stain may comprise keratin biomarkers targeting different keratin forms such as CK-5, CK-8, CK-14, CK-18 (wherein ‘CK’ stands for ‘cytoskeletal keratin’). Self-speaking, the IHC stain may comprise different or additional biomarkers such as p63 and AMACR biomarkers.

According to some examples, the further whole slide image may come from the same tissue sample of the patient, in particular the same tissue section. For instance, the whole slide image and the further whole slide image may relate to consecutive proximal slices of a tissue sample. Further, it is conceivable that the further whole slide image is prepared from a different tissue sample, in particular from a different location in the patient's body. For instance, the whole slide image and the further whole slide image may relate to different biopsies.

Furthermore, according to some examples, generating the further whole slide image may comprise: providing an image processing function configured to simulate image data depicting a tissue slice stained with a different histopathological stain as the histopathological stain of the whole slide image based on image data of whole slide image, and generating the further whole slide image by applying the image processing function on the whole slide image.

The image processing function may be a machine learned function which has been trained according to the above task of simulating a further whole slide image. In particular, the image processing function may be an image-to-image neural network or, more specifically, a convolutional image-to-image neural network. According to some examples, the image processing function may be a generative adversarial network (GAN). According to some examples, the image processing function may be trained based on a whole slide image and a “real” further whole slide image which may be provided as herein described.

The provision of the further whole slide image by ways of the image processing function allows for an automatic generation of the further whole slide image without manual processing steps.

According to some examples, the medical examination may comprise generating radiology image data.

Accordingly, the instructions may comprise an imaging protocol for generating the radiological image data. The imaging protocol may comprise an indication of a body region of the patient to be imaged with an imaging modality, a type of an imaging modality, and/or settings and parameters for controlling the imaging modality as well as for preparing the patient (e.g., in terms of the placement of the patient or an administration of a contrast agent and the like). Further, the instructions may comprise scheduling the medical examination in the healthcare information system, e.g., by allocating a medical imaging modality and, if required, a technician to perform the examination.

The radiological image data may be processed as herein described. In particular, one or more radiological observables may be obtained as herein described.

According to some examples, the medical examination may comprise a laboratory examination, in particular, at least one of a blood test, a flow cytometry test, and/or a gene sequencing test.

The mentioned laboratory tests can provide additional insights in the aggressivity of the cancerous disease and/or the constitution of the patient, which may be helpful in further verifying a treatment option and improving the uncertainty measure.

A blood test can comprise identifying inflammation markers, such as C-reactive protein (CRP)-value or an alkaline phosphatase (AP)-value, an amount of erythrocytes, or tumor markers in a patient's blood sample. Thereby, the inflammatory markers may indicate the presence of inflammation in connection with the cancerous disease and give insights into the disease progression. The same holds true for the AP-value. Likewise, too many or too few erythrocytes may hint at specific issues in connection with the cancerous disease, in particular, issues with the bone marrow. Similarly, tumor markers may further pinpoint the cancerous disease and therewith the available treatment options.

With flow cytometry, physical and chemical characteristics of a population of cells of a sample taken from the patient may be measured. In particular, a so-called FACS-test (FACS=fluorescence-activated cell sorting) may be used. Flow cytometry is useful for diagnosing health disorders, in particular, blood cancers and is therefore effective in further characterizing the health state of the patient. In turn, flow cytometry measurement may be used to verify treatment options and improve the certainty measures.

With gene sequencing or (genome sequencing or cancer genome sequencing), e.g., of a tumor tissue sample of the patient, the specific and unique changes the patient has undergone to develop the cancerous disease can be identified. Based on these changes, a more personalized therapeutic strategy can be defined and the treatment options can be further concretized.

According to an aspect, the method further comprises obtaining supplementary information associated to the patient from a healthcare information system, wherein the prediction function is further configured to derive the treatment response prediction additionally based on supplementary information, and the step of applying comprises additionally applying the prediction function on the supplementary information.

According to some examples, the supplementary information may be information not comprised in the whole slide image. For instance, the supplementary information may comprise further whole slide images as herein described. In particular, the piece of information may relate to non-whole-slide image data. For instance, the supplementary information may comprise radiology image data as herein described. According to further examples, the supplementary information may be non-image data of the patient. According to some examples, the supplementary information may comprise data from a laboratory examination as herein described. According to some examples, the supplementary information comprises structured and/or unstructured natural language text.

According to some examples, the supplementary information comprises one or more of the following elements:
further whole slide images from the same or a different tissue sample/biopsy of the patient, and/or
radiology image data, and/or
data from a laboratory examination (laboratory data), and/or
a prior medical report of the patient, and/or
a-priori knowledge of at least one type of cancerous disease, and/or
a medical guideline applicable for the patient, and/or
an electronic health record of the patient.

According to some examples, the electronic health record may comprise the patient history of the patient including any pre-existing illnesses, comorbidities, risk factors, referral letters, demographic information such as age or gender, and the like.

Obtaining the supplementary information may comprise querying a healthcare information system such as a HIS (hospital information system), a LIS (laboratory information system), an EMR-system (electronic medical record system) and the like for supplementary information of the patient. Such supplementary information may be obtained in the form of one or more EMR-files (electronic medical record-files), for instance. Further, querying healthcare information systems may be based on a patient identifier such as an ID or the patient's name, electronically identifying the patient in the system.

By taking supplementary information into account, an improved treatment response prediction may be realized as the supplementary information may provide additional insights into the cancer the patient is suffering from. In turn, this may help to further narrow down possible treatment options. As the user does not need to consider the supplementary information manually, this reduces the workload of the user and may allow for a more targeted treatment protocol which is better adapted to the particular patient and cancer type.

According to some examples, the method further comprises extracting a supplementary feature vector from the supplementary information, wherein the prediction function is further configured to generate a histopathological feature vector based on the whole slide image and determine the treatment response prediction based on a combined feature vector of the supplementary feature vector and the histopathological feature vector.

According to some examples, the method further comprises providing a plurality of different data processing modules each configured to extract a corresponding observable from a particular data element of the supplementary information, selecting at least one data processing module from the plurality of different data processing modules based on the supplementary information (or the particular data elements comprised in the supplementary information), applying the selected data analysis module on the supplementary information (the particular data element) so as to obtain the corresponding observable, wherein the prediction function is further configured to derive the treatment response prediction additionally based on the corresponding observable(s), and the step of applying comprises additionally applying the prediction function on the corresponding observable.

According to some examples, the corresponding observable(s) may respectively comprise a corresponding feature vector (which may be the supplementary feature or part of the supplementary feature vector), wherein the prediction function is further configured to generate a histopathological feature vector based on the whole slide image and determine the treatment response prediction based on a combined feature vector of the corresponding feature vector(s) and the histopathological feature vector.

The particular data elements may comprise laboratory data, radiology image data, unstructured or unstructured text such as medical reports, and so forth. The data processing modules may comprise dedicated data processing modules such as image or text processing modules. For instance, the data processing modules may comprise a semantic data extraction module and or a radiology image analysis module as herein described. By providing different modules, the method is enabled to process multi-modal data which can render the treatment response prediction more precise.

Essentially the same steps may be applied when processing a piece of information. Specifically, a selection may be performed dependent on the particular piece of information.

According to an aspect, the method further comprises providing a (semantic) data extraction module configured to search an electronic medical record of the patient for supplementary information (or the piece of information), the (semantic) data extraction module preferably comprising a large language model, wherein the step of obtaining comprises accessing the electronic medical record of the patient in the healthcare information system, and applying the data extraction module on the electronic medical record so as to obtain the supplementary information (the piece of information).

The (semantic) data extraction module may generally be configured to identify relevant information for providing a treatment response prediction in a corpus of structured or unstructured textual data related to the patient. The (semantic) data extraction module may be rule based, e.g., by having been programmed to search for a set of predefined keywords. Further, the (sematic) data extraction module may comprise a trained function which has acquired knowledge about inherent syntax and semantics of the human language through training.

A large language model (LLM) is a language model characterized by its large size. In particular, the large language model may be based on a transformer architecture. According to some examples, the large language model may comprise or may be based on models available at the date of filing such as GPT models (e.g., GPT-3.5 and GPT-4), PaLM, LLaMa, LLaMa 2, Falcon, Whisper, and the like.

With the semantic data extraction model, text data can be automatically searched for relevant information. This enables to also leverage unstructured text data which would, otherwise, be difficult to handle. An advantage of large language models is that large language models can efficiently deal with, synonyms, complex semantic relations, and long-range dependencies in input data. Further, transformer networks are capable of processing data in parallel which saves computing resources in inference.

According to an aspect, the supplementary information comprises radiology image data depicting a manifestation of the cancerous disease in the body of the patient.

The radiology image data may comprise one or more medical image data sets. A medical image data set may be a two-dimensional image. Further, the medical image data set may be a three-dimensional image. Further, the medical image may be a four-dimensional image, where there are three spatial and one time-like dimensions. Further, the medical image data set may comprise a plurality of individual medical images.

The medical image data set comprises image data, for example, in the form of a two-or three-dimensional array of pixels or voxels. Such arrays of pixels or voxels may be representative of color, intensity, absorption or other parameters as a function of two or three-dimensional position, and may, for example, be obtained by suitable processing of measurement signals obtained by a medical imaging modality or image scanning facility.

The medical image data set may be a radiology image data set depicting a body part of a patient. Accordingly, it may contain two or three-dimensional image data of the patient's body part. The medical image may be representative of an image volume or a cross-section through the image volume. The patient's body part may be comprised in the image volume.

A medical imaging modality corresponds to a system used to generate or produce medical image data. For example, a medical imaging modality may be a computed tomography system (CT system), a magnetic resonance system (MR system), an angiography (or C-arm X-ray) system, a positron-emission tomography system (PET system) or the like. Specifically, computed tomography is a widely used imaging method and makes use of “hard” X-rays produced and detected by a spatially rotating instrument. The resulting attenuation data (also referred to as raw data) is processed by a computed analytic software producing detailed images of the internal structure of the patient's body parts. The produced sets of images are called CT-scans which may constitute multiple series of sequential images to present the internal anatomical structures in cross sections perpendicular to the axis of the human body. Magnetic Resonance Imaging (MRI), to provide another example, is an advanced medical imaging technique which makes use of the effect magnetic field impacts on movements of protons. In MRI machines, the detectors are antennas, and the signals are analyzed by a computer creating detailed images of the internal structures in any section of the human body.

The medical image may be stored in a standard image format such as the Digital Imaging and Communications in Medicine (DICOM) format and in a memory or computer storage system such as a Picture Archiving and Communication System (PACS), a Radiology Information System (RIS), and the like. Whenever DICOM is mentioned herein, it shall be understood that this refers to the “Digital Imaging and Communications in Medicine” (DICOM) standard, for example according to the DICOM PS3.1 2020c standard (or any later or earlier version of said standard).

The manifestation may be an image of a body part or organ affected by the cancerous disease. As such, the radiology image data may show lesions, tumors, nodules, etc. as manifestations of the disease.

By taking radiology image data into account, orthogonal information regarding the disease may be gathered. Specifically, in contrast to the whole slide image, the radiology image data will show a tumor on a coarser level but in relation to other bodily compartments of the patient such as organs. Further, it might become possible to estimate the overall morphology of the cancerous growth and if and how a primary tumor has spread.

According to an aspect, the method further comprises providing an image analysis module configured to extract a radiological observable from radiology image data, applying the image analysis module on the radiology image data so as to obtain the radiological observable, wherein the prediction function is further configured to derive the treatment response prediction additionally based on the radiological observable, and the step of applying comprises additionally applying the prediction function on the radiological observable.

According to some examples, the radiological observable comprises a radiological feature vector and/or a plurality of different radiological observables may be integrated in a radiological feature vector, wherein the prediction function is further configured to generate a histopathological feature vector based on the whole slide image and determine the treatment response prediction based on a combined feature vector of the radiological feature vector and the histopathological feature vector.

By deriving radiological observables from the radiological image data, relevant features can be automatically extracted which are pertinent for a treatment response prediction. The observables may be learned or predetermined.

According to an aspect, the radiological observable comprises at least one of a tumor burden of the patient in the radiological image data, a visual characteristic of a lesion depicted in the radiological image data, and/or a temporal evolution of a lesion depicted in the radiological image data.

The tumor burden indicates how severely the patient's body or body part is affected by tumors.

For example, the tumor load can be detected by administering suitable contrast agents in a PET-CT examination with subsequent image data analysis by an appropriately configured image analysis module. The PET-CT examination is a combination of positron emission tomography (PET) and computed tomography (CT). PET can visualize metabolic processes in the body. For this purpose, tiny amounts of radioactively labelled substances are administered to the patient. The substances are distributed throughout the body and accumulate in certain tissues, e.g., tumors.

For instance, FDG (F-18 deoxyglucose) can be used as a maker. FDG is a glucose molecule labeled with radioactive fluorine. Since cancer cells have an increased glucose consumption compared to healthy cells, FDG accumulates more in the diseased cells. The different distribution in the body's cells is made visible with the help of the PET camera. Even cancer foci measuring a few millimeters can be detected in this way.

The PET-CT combination device makes it possible to perform computed tomography almost simultaneously. By combining both methods, cell areas with high sugar metabolism activity can be detected and thus provide information about the tumor burden.

A visual characteristic can comprise a size and/or shape and/or morphology and/or contour of a lesion. For instance, the morphology can comprise a level of calcification or solidification of the lesion, while the contour may comprise a level of spiculation or lobulation.

The temporal evolution may comprise a growth or shrinkage of a size of a lesion over time. Further, the temporal evolution may comprise a new formation of a lesion or a vanishing lesion.

The radiological observables relate to values that are difficult for the user to estimate or even quantify. At the same time, those values have proven very valuable to determine a treatment response. Therefore, the automated extraction of such values is particularly beneficial for further supporting the user and coming to a sound treatment response prediction.

According to an aspect, the treatment options comprise at least two different radiotherapy options, the at least two different radiotherapy options differing at least in one of:
a dose distribution,
a dose threshold limit,
a dose rate,
a fractionation,
a usage of proton/photon or electron radiation, and/or
a usage of a co-planar or non-co-planar beam.

In other words, a decision support is offered which allows to decide about the most promising radiotherapy treatment option. The inventors recognized that the tumor characteristics which can be derived from whole slide images can give insights how the tumor would react to different kinds of ionizing radiation. For instance, if the tumor is generally susceptible for ionizing radiation, a lower dose may already be sufficient which has fewer side effects.

According to some examples, the treatment response prediction comprises one or more parameters for a radiotherapy treatment of the patient, wherein the one or more parameters comprise at least one of: a dose distribution, a dose threshold limit, a dose rate, a fractionation, a usage of proton/photon or electrons radiation, and/or a usage of co-planar or non-co-planar beam.

According to an aspect, the one or more treatment option comprises a radiotherapy treatment with a dose rate greater than 40 Gy/sec.

Dose rates greater than 40 Gy/sec typically refer to FLASH radiotherapy. FLASH is a technique involving the delivery of ultra-high dose rate radiation to the target. In preclinical models, FLASH has been shown to reduce radiation-induced toxicity in healthy tissues without compromising the anti-cancer effects of treatment compared to conventional radiation therapy. The FLASH effect designates normal tissue sparing at ultra-high dose rate (>40 Gy/s) compared to conventional dose rate (˜0.1 Gy/s) irradiation while maintaining tumor control and has the potential to improve the therapeutic ratio of radiotherapy. It is noted that non-cancerous cells exposed to ultra-high dose rates of radiotherapy are more likely to be viable than those exposed to conventional dose rates. FLASH could have advantages when referring to organs at risk (OARs) dose limits or any kind of normal tissue toxicity. FLASH could have disadvantages when referring to fractionation regimens and multibeam treatments.

By predicting the treatment response for ultra high dose rates in radiotherapy, the user is provided with an indication if FLASH comes into question for treating the patient. In particular, the combination of information extracted from whole slide images (which can provide a readout for the susceptibility of the tumor cells against FLASH) and radiology image data (which can provide information about the size and location of the tumors) as input data for the prediction has proven to yield a confident result in this regard.

According to an aspect, a system for providing a treatment response prediction for a patient suffering from a cancerous disease comprising an interface unit and a computing unit is provided. The interface unit is configured to obtain a whole slide image of the patient showing a tissue sample relating to the cancerous disease. The computing unit is configured to host a prediction function configured to derive a treatment response prediction for one or more treatment options from whole slide images, to apply the prediction function to the whole slide image so as to generate the treatment response prediction, and to provide the treatment response prediction via the interface unit.

The computing unit may be realized as a data processing system or as a part of a data processing system. Such a data processing system can, for example, comprise a cloud-computing system, a computer network, a computer, a tablet computer, a smartphone and/or the like. The computing unit can comprise hardware and/or software. The hardware can comprise, for example, one or more processors, one or more memories and combinations thereof. The one or more memories may store instructions for carrying out the method steps according to embodiments of the present invention. The hardware can be configurable by the software and/or be operable by the software. Generally, all units, sub-units or modules may at least temporarily be in data exchange with each other, e.g., via a network connection or respective interfaces. Consequently, individual units may be located apart from each other.

The interface unit may comprise an interface for data exchange with a local server or a central web server via internet connection for receiving the whole slide images and/or prediction functions. The interface unit may be further adapted to interface with one or more users of the system, e.g., by displaying the result of the processing by the computing unit to the user (e.g., in a graphical user interface) or by allowing the user to adjust parameters for image processing or visualization, for selecting whole-slide images for processing, and/or reviewing treatment options.

According to other aspects, embodiments of the present invention further relate to a digital pathology image analysis system comprising at least one of the above systems and a healthcare information system configured to acquire, store and/or forward at least whole slide images and, optionally, also other data such as the supplementary information. Thereby, the interface unit is configured to receive whole slide images form the digital pathology image system.

According to some examples, the healthcare information system comprises one or more archive stations for storing whole slide images and/or supplementary information which archive stations may be realized as a cloud storage or as a local or spread storage. Further, the healthcare information system may comprise one or more imaging modalities, such as a slide scanning apparatus or radiology imaging modalities or the like.

According to an aspect, a computer-implemented method for providing a prediction function is provided. The method comprises a plurality of steps. A first step is directed to provide a machine-learnable prediction function configured for providing, based on a whole slide image, a treatment response prediction for a treatment option for treating a cancerous disease. A further step is directed to provide input training data comprising a whole slide showing a tissue sample relating to the cancerous disease of a patient. A further step is directed to provide training output data comprising a treatment response of the patient for the at least one treatment option. A further step is directed to input the training input data into the prediction function to obtain an intermediate treatment response prediction. A further step is directed to compare the intermediate treatment response prediction with the output training data. A further step is directed to adjust the prediction function based on the step of comparing. A further step is directed to provide the adjusted prediction function.

According to another aspect, a training system for providing a prediction function for providing a treatment response prediction. The system comprises an interface embodied for receiving the prediction function and the input and output training data. The system further comprises a computing unit configured to run the prediction function. The computing unit is further configured to carry out the training steps according to the method for providing a prediction function as herein described.

According to another aspect, an embodiment of the present invention is directed to a non-transitory computer program product comprising program elements which induce a computing unit of a system to perform the steps according to one or more of the method aspects and examples herein described, when the program elements are loaded into a memory of the computing unit.

According to another aspect, an embodiment of the present invention is directed to a non-transitory computer-readable medium on which program elements are stored that are readable and executable by a computing unit of a system according to one or more method aspects and examples herein described, when the program elements are executed by the computing unit.

The realization of one or more embodiments of the present invention by a computer program product and/or a computer-readable medium has the advantage that already existing providing systems can be easily adapted by software updates in order to work as proposed by one or more embodiments of the present invention.

The computer program product can be, for example, a computer program or comprise another element next to the computer program as such. This other element can be hardware, e.g., a memory device, on which the computer program is stored, a hardware key for using the computer program and the like, and/or software, e.g., a documentation or a software key for using the computer program. The computer program product may further comprise development material, a runtime system and/or databases or libraries. The computer program product may be distributed among several computer instances.

Independent of the grammatical term usage, individuals with male, female or other gender identities are included within the term.

### BRIEF DESCRIPTION OF THE DRAWINGS

Characteristics, features and advantages of the above-described invention, as well as the manner they are achieved, become clearer and more understandable in the light of the following description of embodiments, which will be described in detail with respect to the figures. This following description does not limit the present invention on the contained embodiments. Same components, parts or steps can be labeled with the same reference signs in different figures. In general, the figures are not drawn to scale. In the following:

FIG. 1 schematically depicts a system for providing a treatment response prediction according to an embodiment,

FIG. 2 schematically depicts a method for providing a treatment response prediction according to an embodiment,

FIG. 3 schematically depicts data flows in a method for providing a treatment response prediction according to an embodiment,

FIG. 4 schematically depicts a method for providing a treatment response prediction according to a further embodiment,

FIG. 5 schematically depicts data flows in a method for providing a treatment response prediction according to a further embodiment,

FIG. 6 schematically depicts a method for providing a treatment response prediction according to a further embodiment,

FIG. 7 schematically depicts data flows in a method for providing a treatment response prediction according to a further embodiment,

FIG. 8 schematically depicts a prediction function for predicting a treatment response according to an embodiment,

FIG. 9 schematically depicts a method for providing a prediction function for predicting a treatment response according to an embodiment, and

FIG. 10 schematically depicts a system for providing a prediction function for predicting a treatment response according to an embodiment.

### DETAILED DESCRIPTION

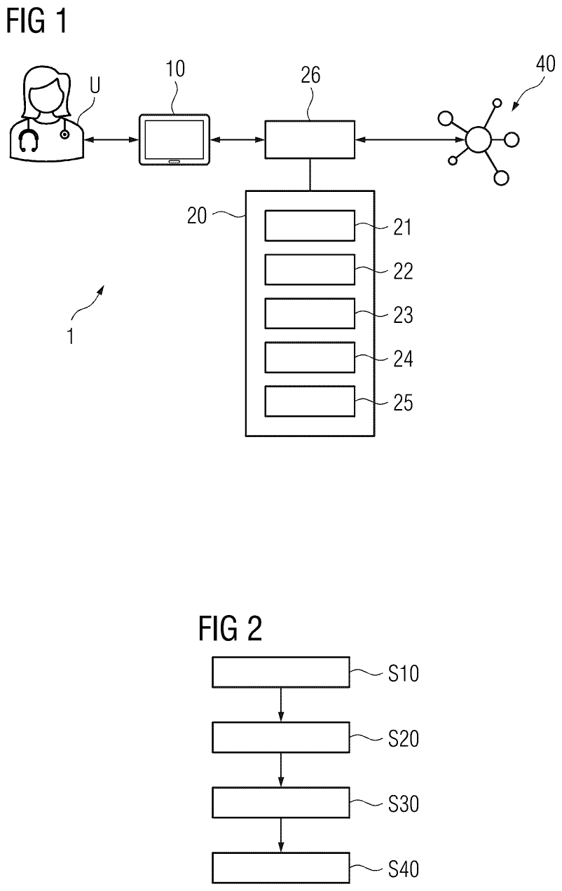

FIG. 1 depicts a system 1 for providing a treatment response prediction TRP in connection with a whole slide image WSI to a user U. In this regard, system 1 is adapted to perform the methods according to one or more embodiments, e.g., as further described with reference to FIGS. 2 to 7. Further, system 1 may be configured to host, provide and/or apply a prediction function PF as shown in

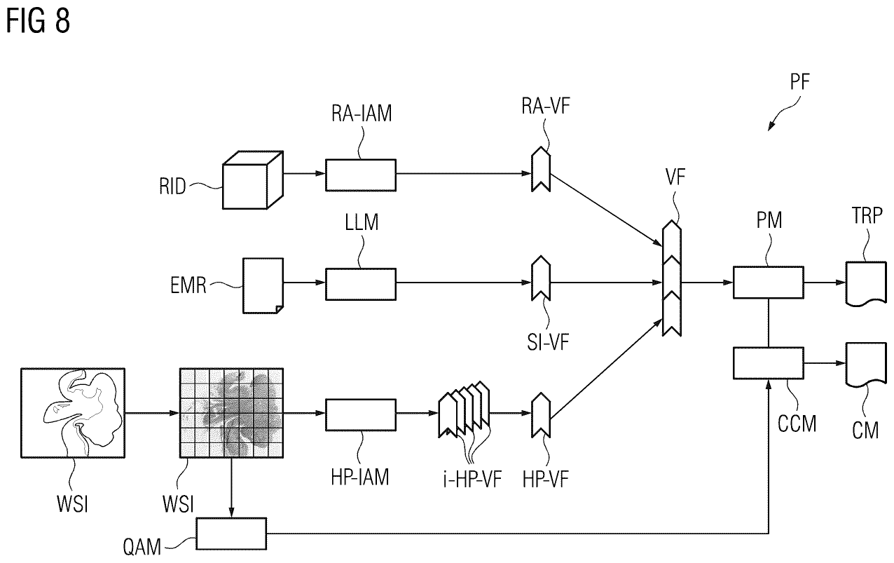

FIG. 8 which has been adapted to derive a treatment response prediction TRP from whole slide images WSI and, optionally, additional, supplementary information SI.

The user U of system 1 (in the sense of an operator controlling the system 1), according to some examples, may generally relate to a healthcare professional such as a physician, clinician, technician, pathologist, radiologist and so forth.

System 1 comprises a user interface 10 (as part of the interface unit) and a processing system 20 (as part of the computing unit). Further, system 1 may comprise or be connected to a medical information system 40. The medical information system 40 may generally be configured for acquiring and/or storing and/or forwarding whole slide images WSI, F-WSI and supplementary information SI. For instance, medical information system 40 may comprise one or more archive/review station (not shown) for whole slide images WSI. The archive/review stations may be embodied by one or more databases. In particular, the archive/review stations may be realized in the form of one or more cloud storage modules. Alternatively, the archive/review stations may be realized as a local or spread storage, e.g., as a PACS (Picture Archiving and Communication System). According to some examples, medical information system 40 may also comprise one or more medical imaging modalities (not shown) for generating whole slide images WSI, such as a slide scanner, but also radiology imaging modalities such as computed tomography systems, magnetic resonance systems, angiography (or C-arm X-ray) systems, positron-emission tomography systems, mammography systems, X-ray systems, or the like.

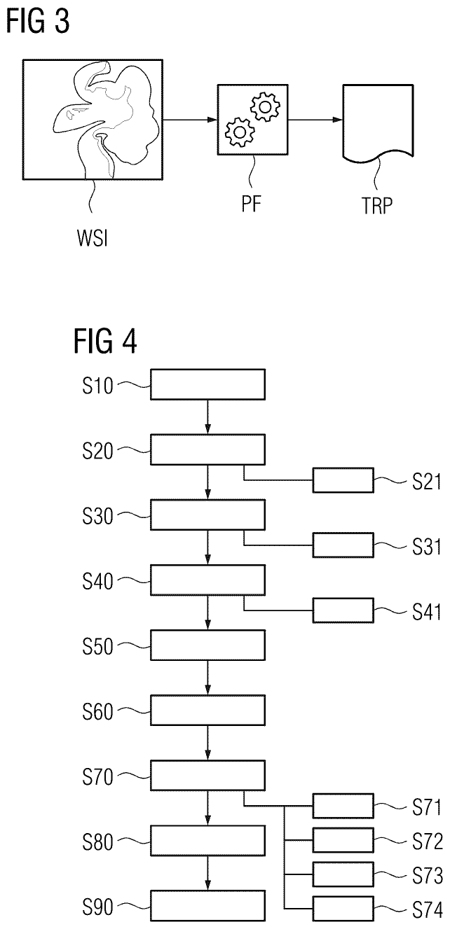

FIGS. 3, 5, and 7 depict schematic representations of whole slide images WSI. In general, a whole slide image WSI, F-WSI depicts tissue slices prepared from tissue samples of a patient. The slices may be prepared by cutting tissue samples into micrometer-thick slices. The whole slide image WSI depicts the tissue slices in microscopic observation. Accordingly, a tissue slice can show the fine tissue structure of the tissue sample and in particular the cell structure or the cells contained in the tissue sample.

In general, a whole slide image will show different tissue types, such as healthy tissue with healthy cells, cancerous tissue with cancerous cells and others, such as amorphous tissue or necrotic tissue. In particular, the cancerous cells may indicate how the tumor would respond to one or more therapy options. This is because the morphology of the cells and the cancerous regions provides indications as to how aggressive the tumor is and as to how susceptible it is vis-à-vis various treatment options. Besides, also the healthy cells may be relevant for choosing the right treatment option, as it may be derived from the morphology of healthy tissue how it would suffer under a particular treatment option and, thus, what would be the most likely side effects of the treatment option.

Whole slide images WSI may depict tissue stained with a particular histopathological stain in order to highlight features in the tissue slides for inspection. The most common stain is the H&E (hematoxylin and eosin) stain. Accordingly, the whole slide image WSI will generally be based on a H&E staining. Furthermore, also different stains may be considered, for instance, in the form of further whole slide images F-WSI of the patient. These further whole slide images F-WSI may be part of the supplementary information SI which may be retrieved for safeguarding a treatment response prediction TRP.

Supplementary information SI (or associate data) may be any data providing additional information relating to the patient and/or the whole slide image WSI. The supplementary information SI may comprise image data such as further whole slide images F-WSI of the patient which were, for instance, acquired at a different point in time than the whole slide image WSI and/or were prepared differently, in particular, using a different histopathological stain. Further, image data may comprise radiology image data RID which was acquired using a radiology imaging modality. Further the supplementary information SI may comprise non-image data or data with mixed-type contents comprising medical images and non-image contents such as text. Non-image data may relate to non-image examination results such as lab data, vital signs records (comprising, e.g., ECG data, blood pressure values, ventilation parameters, oxygen saturation levels) and so forth. Moreover, the supplementary information SI may comprise structured and unstructured medical text reports relating to prior examinations or the current examination of the patient. Further, non-image data may comprise personal information of the patient such as gender, age, weight, insurance details, and so forth.

The supplementary information SI may be available in the form of one or more electronic medical records EMR of the patient. The supplementary information SI may be stored in the healthcare information system 40. For instance, the supplementary information SI may be stored in dedicated databases of the healthcare information system 40 such as laboratory information system (LIS) or an electronic health/medical record database.

Radiology image data RID may be three-dimensional image data sets acquired, for instance, using an X-ray system, a computed tomography system or a magnetic resonance imaging system or other systems. The image information may be encoded in a three-dimensional array of m times n times p voxels. Radiology image data sets may include a plurality of image slices which are stacked in a stacking direction to span the image volume covered by the radiology image data RID.

Further, radiology image data RID may comprise two-dimensional medical image data with the image information being encoded in an array of m times n pixels. According to some examples, these two-dimensional medical images may have been extracted from three-dimensional radiology image data RID.

Radiology image data RID as well as whole slide images WSI, F-WSI may be formatted according to the DICOM format. DICOM (=Digital Imaging and Communications in Medicine) is an open standard for the communication and management of medical imaging information and related data in healthcare informatics. DICOM may be used for storing and transmitting medical images and associated information enabling the integration of medical imaging devices such as scanners, servers, workstations, printers, network hardware, and picture archiving and communication systems (PACS). It is widely adopted by clinical syndicates, hospitals, as well as for smaller applications like doctors' offices or practices. A DICOM data object consists of a number of attributes, including items such as the patient's name, ID, etc., and also special attributes containing the image pixel data and metadata extracted from the image data.

User interface 10 may comprise a display unit and an input unit. User interface 10 may be embodied by a mobile device such as a smartphone or tablet computer. Further, user interface 10 may be embodied as a workstation in the form of a desktop PC or laptop. The input unit may be integrated in the display unit, e.g., in the form of a touch screen. As an alternative or in addition to that, the input unit may comprise a keyboard, a mouse or a digital pen and any combination thereof. The display unit may be configured for displaying a representation of the whole slide image WSI, excerpts from the supplementary information SI, one or more treatment response predictions TRP, and/or certainty measures CM.

User interface 10 may further comprise an interface computing unit configured to execute at least one software component for serving the display unit and the input unit in order to provide a graphical user interface for allowing the user U to select a target patient's case to be reviewed and making various inputs. In addition, the interface computing unit may be configured to communicate with medical information system 40 or processing system 20 for receiving the whole slide image WSI and any supplementary information SI. The user U may activate the software component via user interface 10 and may acquire the software component, e.g., by downloading it from an internet application store. According to an example, the software component may also be a client-server computer program in the form of a web application running in a web browser. The interface computing unit may be a general processor, central processing unit, control processor, graphics processing unit, digital signal processor, three-dimensional rendering processor, image processor, application specific integrated circuit, field programmable gate array, digital circuit, analog circuit, combinations thereof, or other now known devices for processing image data. User interface 10 may also be embodied as a client.

Processing system 20 may comprise sub-units 21-25 configured to process the whole slide image WSI, F-WSI and supplementary information SI in order to provide a treatment response prediction TRP for specific treatment options coming into question for treating the cancerous disease of the patient.

Processing system 20 may be a processor. The processor may be a general processor, central processing unit, control processor, graphics processing unit, digital signal processor, three-dimensional rendering processor, image processor, application specific integrated circuit, field programmable gate array, digital circuit, analog circuit, combinations thereof, or other now known device for processing image data. The processor may be single device or multiple devices operating in serial, parallel, or separately. The processor may be a main processor of a computer, such as a laptop or desktop computer, or may be a processor for handling some tasks in a larger system, such as in the medical information system or the server. The processor is configured by instructions, design, hardware, and/or software to perform the steps discussed herein. The processing system 20 may be comprised in the user interface 10. Alternatively, processing system 20 may comprise a real or virtual group of computers like a so called ‘cluster’ or ‘cloud’. Such server system may be a central server, e.g., a cloud server, or a local server, e.g., located on a hospital or radiology site. Further, processing system 20 may comprise a memory such as a RAM for temporally loading the medical image data sets MIDS. According to some examples, such memory may as well be comprised in user interface 10.

Sub-unit 21 is a data retrieval unit. It is configured to access and search the medical information system 40 for supplementary information SI and/or pieces of information PI which could render a prediction more conclusive. Specifically, sub-unit 21 may be configured to formulate search queries and parse them to the medical information system 40. According to some examples, the search queries may be based on an electronic patient identifier of the patient in the medical information system 40.

Sub-unit 22 may be conceived as a data analysis unit. Sub-unit 22 is configured to derive observables RA-VF, HP-VF, SI-VF from the available data WSI, F-WSI, EMR, RID. Specifically, sub-unit 22 may be configured to extract a histopathology feature vector HP-VF from whole slide images WSI, F-WSI, a radiology feature vector RA-VF from radiology image data RID and/or a data feature vector SI-VF from an electronic medical record EMR of the patient. To this end, sub-unit 22 may be configured to host and run a plurality of dedicated processing modules RA-IAM, HP-IAM, LLM of the prediction function PF.

Sub-unit 23 may be conceived as prediction unit. Sub-unit 23 may be configured to take the observables provided by the data analysis unit 22 and classify these according to one or more treatment options. On that basis, a response of the patient to the treatment option may be predicted and provided as treatment response prediction TRP. Sub-unit 23 may be configured to host and run a prediction module PM of the prediction function PF.

Sub-unit 24 may be conceived as a certainty calculation unit. It may be configured to calculate a certainty measure CM which indicates how confident a treatment response prediction TRP is. It may further be configured to determine additional data which could render a treatment response prediction TRP more confident. This additional data is also designated as piece of information PI. The piece of information PI may be a part of the supplementary information SI available for the patient. Further, the piece of information PI may be a hypothetical, not yet existing information. Sub-unit 24 may be configured to host and run a correspondingly configured certainty calculation module CCM of the prediction function PF.

Sub-unit 25 may be configured as an input/output unit. Sub-unit 25 may be configured to provide a treatment response prediction TRP for displaying to the user U via the user interface 10. Moreover, sub-unit 25 may be configured to check if the piece of information PI as determined by sub-unit 24 is available in healthcare information system 40. Further, sub-unit 25 may be configured to generate an instruction INS directed to (newly) generate a piece of information PI, wherein the instruction INS may be directed either to the user U as human interpretable output or to an appropriate modality for generating the piece of information PI as machine readable control signals. Further, sub-unit 25 may be configured to output the instruction INS either to the user U via user interface 10 or to the appropriate modality.

The designation of the distinct sub-units 21-25 is to be construed by way of example and not as a limitation. Accordingly, sub-units 21-25 may be integrated to form one single unit (e.g., in the form of “the computing unit”) or can be embodied by computer code segments configured to execute the corresponding method steps running on a processor or the like of processing system 20. The same holds true with respect to the interface computing unit. Each sub-unit 21-25 and the interface computing unit may be individually connected to other sub-units and/or other components of the system 1 where data exchange is needed to perform the method steps.

Processing system 20 and the interface computing unit(s) together may constitute the computing unit of the system 1. Of note, the layout of this computing unit, i.e., the physical distribution of the interface computing unit and sub-units 21-25 is, in principle, arbitrary. Specifically, processing system 20 may also be integrated in user interface 10. As already mentioned, processing system 20 may alternatively be embodied as a server system, e.g., a cloud server, or a local server, e.g., located on a hospital or radiology site. According to such implementation, user interface 10 could be designated as a “frontend” or “client” facing the user U, while processing system 20 could then be conceived as a “backend” or server. Communication between user interface 10 and processing system 20 may be carried out using the https-protocol, for instance. The computational power of the system may be distributed between the server and the client (i.e., user interface 10). In a “thin client” system, the majority of the computational capabilities exists at the server. In a “thick client” system, more of the computational capabilities, and possibly data, exist on the client.

Individual components of system 1 may be at least temporarily connected to each other for data transfer and/or exchange. User interface 10 communicates with processing system 20 via (data) interface 26 to exchange, e.g., whole slide images WSI, treatment response predictions TRP, or instructions INS for generating missing information PI. For example, processing system 20 may be activated on a request-base, wherein the request is sent by user interface 10. Further, processing system 20 may communicate with medical information system 40 in order to retrieve a target patient's case. As an alternative or in addition to that, user interface 10 may communicate with medical information system 40 directly. Medical information system 40 may likewise be activated on a request-base, wherein the request is sent by processing system 20 and/or user interface 10. Data interface 26 for data exchange may be realized as hardware- or software-interface, e.g., a PCI-bus, USB or fire-wire. Data transfer may be realized using a network connection. The network may be realized as local area network (LAN), e.g., an intranet or a wide area network (WAN). Network connection is preferably wireless, e.g., as wireless LAN (WLAN or Wi-Fi). Further, the network may comprise a combination of different network examples. Interface 26 for data exchange together with the components for interfacing with the user U be regarded as constituting an interface unit of system 1.

FIG. 2 depicts a method for providing a text summary TS according to an embodiment. Corresponding data streams are illustrated in FIG. 3. The method comprises several steps. The order of the steps does not necessarily correspond to the numbering of the steps but may also vary between different embodiments of the present invention. Further, individual steps or a sequence of steps may be repeated.

At step S10, a whole slide image WSI is provided. This may involve selecting the whole slide image WSI from a plurality of cases, e.g., stored in the medical information system 40. The selection may be performed manually by the user U, e.g., by selecting appropriate image data in a graphical user interface running in the user interface 10. Alternatively, the whole slide image WSI may be provided to the computing unit by the user U by way of uploading the whole slide image WSI to the processing system 20.

The whole slide image WSI is prepared from a tissue sample of the patient's cancerous disease and may be stained with an H&E stain.

At step S20, the prediction function PF is provided. The prediction function PF is configured to process whole slide images WSI and, optionally, supplementary information SI such as radiology image data RID or an electronic medical record EMR of the patient, in order to derive cues how the patient or the cancer the patient is suffering from would respond to a plurality of different treatment options. To this end, the prediction function PF may comprise a plurality of different data processing modules RA-IAM, HP-IAM, LLM which have respectively been configured to extract characteristics from the different input data WSI, F-WSI, RID, EMR. The characteristics may also be referred to as observables for the respective input data. The observables may respectively comprise a plurality of features. Thus, each observable may have the form of a feature vector HP-VF, RA-VF, SI-VF. The feature vectors HP-VF, RA-VF, SI-VF may be mapped to one or more treatment options by a classification or prediction module PM. In other words, a mapping is performed indicating how well the feature vector(s) fit to the one or more treatment options. Based on the mapping, the treatment response prediction TRP may be calculated.

The treatment options may be pre-defined and relate to different treatments typically administrated to patients suffering from the cancerous disease. The treatment options may comprise radiotherapy treatment options, chemotherapy treatment options, surgical treatment options, immunotherapy treatment options or any combinations of the aforesaid. Further different treatment options may set out different parameters of the same kind of treatment such as medication doses and/or radiotherapy parameters.

Further details regarding the prediction function PF will be described in connection with FIG. 8. The prediction function PF may be provided by holding it available in a memory of the processing system 20, for instance, as executable computer code.

At step S30, the prediction function PF is applied on the whole slide image WSI so as to automatically process the whole slide image WSI in order to provide the treatment response prediction TRP as a form of a diagnosis of the patient. Specifically, this may involve extracting a histopathological feature vector HP-VF from the whole slide image WSI and classifying the feature vector HP-VF according to at least one, preferably a plurality of treatment options. The classification or mapping may indicate how well the histopathological feature vector HP-VF “fits” to individual treatment options. In turn, this “fit” may quantify how likely the corresponding treatment option will be successful for the patient and may form the basis for the treatment response prediction TRP.

At step S40, the treatment response prediction TRP may be provided. This may involve showing the treatment response prediction TRP to the user U in the user interface 10, e.g., in a suitable graphical user interface.

According to some examples, the treatment response prediction TRP may comprise a recommendation for a certain treatment option, e.g., the one with the highest success rate. Specifically, the treatment response prediction TRP may comprise a recommendation for a radiotherapy treatment involving a certain dose distribution, dose threshold limit, a dose rate, a fractionation, a usage of proton/photon or electron radiation, and/or a usage of a co-planar or non-co-planar beam. Moreover, the treatment response prediction TRP may comprise a recommendation for a radiotherapy treatment involving FLASH.

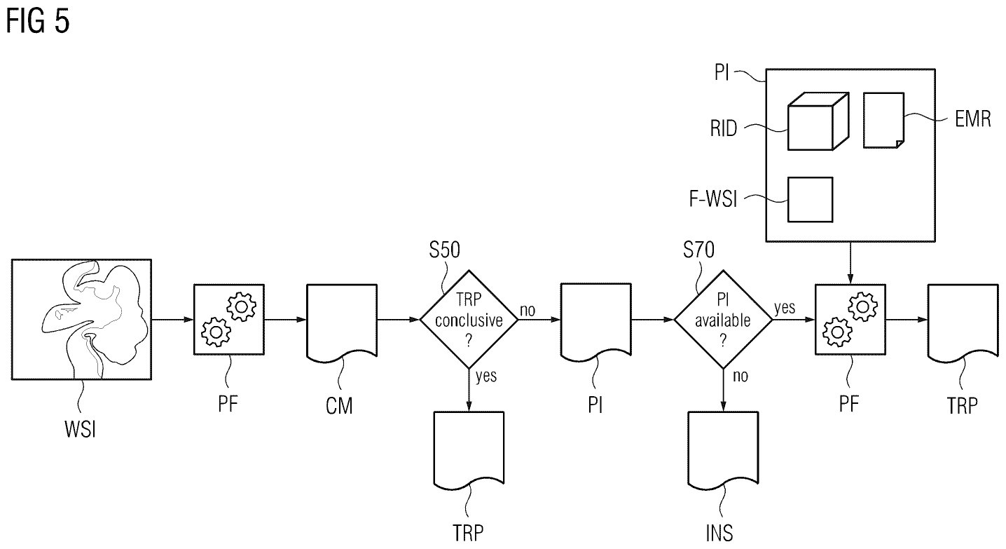

FIG. 4 depicts a method for providing a treatment response prediction TRP according to an embodiment. Corresponding data streams are illustrated in FIG. 5. The method comprises several steps. The order of the steps does not necessarily correspond to the numbering of the steps but may also vary between different embodiments of the present invention. Further, individual steps or a sequence of steps may be repeated. Specifically, the method according to FIGS. 4 and 5 is directed to an optional workflow involving the calculation and usage of a certainty measure CM. Method steps denoted with like reference numerals in FIGS. 2 and 4 correspond to the same method steps.

A certainty measure CM may indicate how confident a certain treatment response prediction TRP is. For example, certainty measures CM may be real numbers selected from an interval from 0 to 1. As will be further explained in connection with FIG. 8, certainty measures CM may be provided by a dedicated certainty calculation module CCM.

At step S21, a certainty calculation module CCM is provided. Step S21 may be an optional sub-step of step S20.

The certainty calculation module CCM may be part of the prediction function PF. The certainty calculation module CCM may be configured to derive a certainty measure CM based on the processing of the actual prediction module PM of the prediction function. Further, the certainty calculation module CCM may be configured to base the certainty measure CM on the quality of the input data, in particular, the whole slide image WSI. To this end, the prediction function may comprise a quality assessment module QAM configured to determine a quality of the input whole slide image WSI.

At step S31, the certainty calculation module CCM is applied. Step S31 may be an optional sub-step of step S30. This may mean that the certainty calculation module CCM is applied to the input and/or output data, i.e., the whole slide image WSI and any supplementary information SI and/or the treatment response prediction, and/or any intermediate processing result such as feature vectors RA-VF, SI-VF, HP-VF and/or outputs of individual layers of the image or data analysis modules RA-IAM, HP-IAM, LLM and/or the prediction module PM.

At step S41, the certainty measure CM is provided. Step S41 may be an optional sub-step of step S40. At step S41, the certainty measure CM may be provided to the user U via user interface 10. Further, the certainty measure CM may be provided for further automated processing steps.

One of the automated further processing steps S50 may involve determining if the treatment response prediction TRP is conclusive or, in other words, certain enough. To this end, the certainty measure CM may be compared to a pre-defined threshold. If the certainty measure CM is below the threshold, the treatment response prediction TRP may be considered as non-conclusive.

That followed, it may optionally be determined at optional step S60 what (hypothetical) piece of information PI could be useful to improve the certainty measure CM. Systematically, this may be derived from the processing of the certainty calculation module CCM at step S31. This is because the certainty calculation module CCM is configured to observe the individual processing steps of the prediction function PF and, thus, is capable of identifying which part of the processing is underperforming.

For instance, if the whole slide image WSI is not of sufficient quality, a further whole slide image F-WSI may be identified as potentially helpful for ascertaining the treatment response prediction TRP. Moreover, if the treatment response prediction TRP is undecided between two treatment options, it may be derived which kind of non-image information may be suited to come to a clearer prediction. This may be counter-indications for a certain treatment derivable from the electronic medical record EMR of the patient or demographic information about the patient such as age, gender, co-morbidities, or already administered therapy forms. In addition, radiology image data RID may be deemed helpful at step S60 to improve the confidence measure CM. This is because radiology image data RID may, for instance, give insights into how much a tumor has already spread.

At optional step S70, it may be determined if the piece of information PI determined at step S60 is already available in the healthcare information system 40. To this end, the healthcare information system 40 may be accessed and queried for the electronic medical record EMR of the patient. That followed, the electronic medical record EMR may be searched for the piece of information PI. According to some examples, this may comprise applying an appropriately configured data analysis module LLM to the electronic medical record EMR. For instance, the data analysis module LLM may comprise a large language module as herein described.

If the piece of information PI is already available, the piece of information PI may be pulled from the healthcare information system 40 at step S71.

If the piece of information PI is not available, a corresponding notification may be provided to the user U at step S72. With that, a user U can initiate steps to gather the piece of information PI by herself.

As an alternative, instructions INS to generate the piece of information PI may be automatically generated as a form of further assistance. The instructions INS may be directed to a user U which enable the user U to obtain the piece of information PI. For instance, this may involve protocols for preparing a further whole slide image F-WSI or imaging protocols for a radiology examination of the patient.

Further, the instructions INS may directly comprise machine readable instructions which can directly be used to control devices in the healthcare information system 40. For instance, such devices may comprise medical imaging modalities or scheduling systems for performing scans or preparing whole slide images WSI.

According to some examples, such instructions INS may be generated by a large language model.

At step S74, the instructions INS may be provided. This may involve inputting the instructions INS in corresponding devices in the healthcare information system 40 for controlling these devices. Further, this may involve outputting the instructions INS to the user U via the user interface 10.

At optional step $80, any piece of information PI obtained may be processed by inputting it in the prediction function PF so as to obtain an updated treatment response prediction TRP and, optionally, an updated certainty measure CM.

That followed, at optional step $90, the updated treatment response prediction TRP and/or the updated certainty measure CM may be provided substantially as described in connection with steps S40 and step S41.

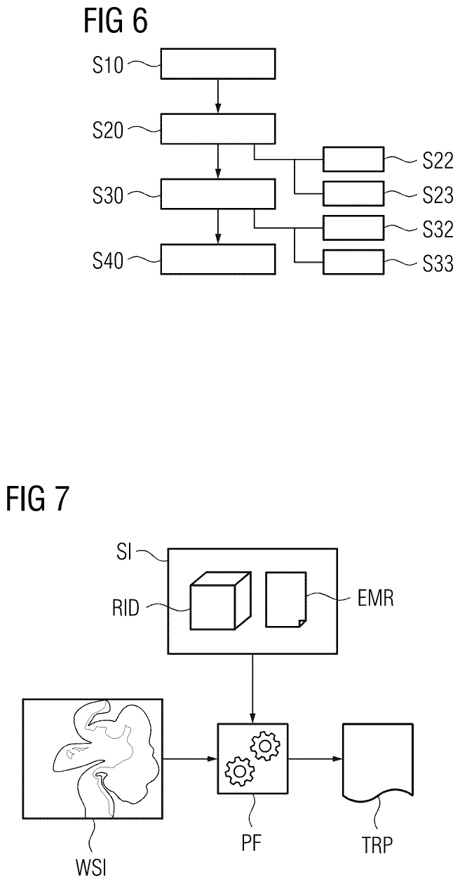

FIG. 6 depicts a method for providing a treatment response prediction TRP according to an embodiment. Corresponding data streams are illustrated in FIG. 7. The method comprises several steps. The order of the steps does not necessarily correspond to the numbering of the steps but may also vary between different embodiments of the present invention. Further, individual steps or a sequence of steps may be repeated. Specifically, the method according to FIGS. 6 and 7 is directed to an optional workflow involving the usage of supplementary information SI for generating the treatment response prediction TRP. Method steps denoted with like reference numerals in FIGS. 2 and 6 correspond to the same method steps. Further, the method steps of FIG. 6 may be combined with the method steps of FIG. 4.

Supplementary information SI may be obtained to be processed by the prediction function PF alongside the whole slide image WSI in an optional sub-step of step S10. The supplementary information SI may be obtained by querying the medical information system 40, e.g., based on an electronic patient identifier of the patient in the medical information system 40.

To process the different kinds of supplementary information SI, the prediction function PF may comprise dedicated processing modules LLM, RA-IAM besides the image analysis module HP-IAM for processing histopathology whole slide images WSI, F-WSI. The modules may respectively be configured to extract observables in the form of feature vectors RA-VF, HP-VF, SI-VF which may be concatenated and mapped to different treatment options in the prediction module PM. The different processing modules may be selected and applied based on the content of the supplementary information SI.

For instance, the processing modules may comprise a data extraction module LLM which may be provided at step S22. The data extraction module LLM may be configured to find and extract information sought for from structured or unstructured text data such as medical reports. To this end, the data extraction module LLM may be a semantic data extraction module, in particular, a large language model.

Further, the processing modules may comprise an image analysis module RA-IAM for extracting relevant observables from radiology image data RID. The radiology image analysis module RA-IAM may be provided at step S23.

At step S32, the radiology image analysis module RA-IAM is applied to radiology image data RID in order to obtain a corresponding radiological observable RA-VF. The radiological observable RA-VF may have the form of a feature vector RA-VF.

Similarly, at step S33, the data extraction module LLM is applied to the electronic medical record EMR of the patient so as to obtain a corresponding data observable SI-VF. The data observable SI-VF may have the form of a feature vector SI-VF.

That followed, any data observables SI-VF, RA-VF thus obtained may be input in the prediction module PM of the prediction function PF together with histopathology observables HP-VF derived from whole slide images WSI, F-WSI.

In FIG. 8, a schematic representation of a prediction function PF according to an embodiment is shown.

As shown in FIG. 8, the prediction function PF may comprise three branches: a branch for processing whole slide images WSI, F-WSI with a histopathology image analysis module HP-IAM, a branch for processing radiology image data RID with a radiology image processing module RA-IMA, and a branch for processing electronic medical records EMR (in particular, non-image data such as structured or unstructured text) with a data extraction module LLM. The “radiology branch” and the “EMR-branch” are optional.

The respective processing modules RA-IAM, HP-IAM, LLM in the individual branches may be configured to extract observables from the corresponding input data which are relevant for predicting the treatment response of the patient. The observables may respectively have the form of a feature vector RA-VF, HP-VF, SI-VF. The feature vectors RA-VF, HP-VF, SI-VF may be learned by machine learning techniques and/or may be pre-defined. The latter may come into question where it is already clear that a certain information is particularly relevant, such as age or gender (in the case of the electronic medical record EMR) or size and distribution of lesion (in the case of the radiology image data RID).

As for the histopathology image analysis module HP-IAM, there are various implementations available for extracting feature vectors HP-VF from whole slide images F-WSI, WSI. For instance, it would be possible to employ a CNN. For instance, a ResNet, in particular a ResNet-18 or a ResNet-50 may be used to convert image data of whole slide images WSI, F-WSI into feature vectors HP-VF.

To enable efficient processing, each whole slide image WSI, WSI-F may be divided into N tiles or patches. For each of the tiles, an intermediate feature vector i-HP-VF may be extracted which may be averaged to obtain the final feature vector HP-VF for the respective whole slide image WSI, F-WSI.

Also regarding the radiology image analysis module RA-IAM, there is a plurality of established options to derive feature vectors RA-VF from radiology image data RID. Preferably, the radiology image analysis module RA-IAM also comprises a ResNet, in particular, a ResNet-50. The radiology image analysis module RA-IAM, may be trained specifically for the prediction function PF. Alternatively or additionally, the radiology image analysis module RA-IAM may comprise pre-configured algorithms which are not specific to the prediction function PF (i.e., which are trained outside of the prediction function PF) and which may generally be used in reading radiology images such as lesion detection and quantification algorithms. The results of these pre-configured algorithms may also be fed into the radiological feature vector RA-VF.

The data extraction module LLM may comprise a transformer architecture. In particular, the data extraction module LLM may comprise a large language module LLM. A transformer network is a neural network architecture generally comprising an encoder, a decoder or both an encoder and decoder. In some instances, the encoders and/or decoders are composed of several corresponding encoding layers and decoding layers, respectively. Within each encoding and decoding layer is an attention mechanism. The attention mechanism, sometimes called self-attention, relates data items (such as words) within a series of data items to other data items within the series. The self-attention mechanism for instance allows the model to examine a group of words within a sentence and determine the relative importance other groups of words within that sentence have to the word being examined.

The encoder, in particular, may be configured to transform the input (text) into a numerical representation. The numerical representation may comprise a vector per input token (e.g., per word). The encoder may be configured to implement an attention mechanism so that each vector of a token is affected by the other tokens in the input. In particular, the encoder may be configured such that the representations resolve the desired output of the transformer network.

The decoder, in particular, may be configured to transform an input into a sequence of output tokens. In particular, the decoder may be configured to implement a masked self-attention mechanism so that each vector of a token is affected only by the other tokens to one side of a sequence. Further, the decoder may be auto-regressive meaning in that intermediate results (such as a previously predicted sequence of tokens) are fed back.

According to some examples, the output of the encoder is input into the decoder. Further, the transformer network may comprise a classification module or unit configured to map the output of the encoder or decoder to a set of learned outputs such as the desired feature vector SI-VF.

Training of a transformer model according to some examples may happen in two stages, a pretraining and a fine-tuning stage. In the pretraining stage, a transformer model may be trained on a large corpus of data to learn the underlying semantics of the problem. Such pre-trained transformer models are available for different languages. For certain applications described herein, the fine-tuning may comprise further training the transformer network with medical texts with expert annotated meanings and/or medical ontologies such as RADLEX and/or SNOMED. With the latter, in particular, the transformer model according to some examples may learn typical relations and synonyms of medical expressions.

For a review on transformer networks, reference is made to Vaswani et al., “Attention Is All You Need”, in arXiv: 1706.03762, Jun. 12, 2017, the contents of which are herein included by reference in their entirety.

According to some examples, data extraction module LLM is configured to derive a number of predefined features SI-VF from the electronic medical record EMR, which are to be added to the feature vector(s) extracted from the whole slide images WSI, F-WSI and/or the radiology image data RID. The predefined features SI-VF may be predetermined influencing factors which would affect the decision making of a human user U regarding one or more treatment options, such as the age, gender, relevant lab values (such as PSA in case of prostate cancer), co-morbidities, already performed therapies and their success, metastasis, etc.

The individual feature vectors RA-VF, HP-VF, SI-VF may be concatenated to form a combined feature vector VF. The combined feature vector VF may then be input in a prediction module PM. For the prediction module PM, a regressor model can be used. The prediction module PM may output the prediction for a treatment response TRP for one or more treatment options. The treatment options may be pre-configured, for instance, based on the training data.

Optionally, the prediction function PF may comprise modules for determining a certainty measure CM for the treatment response prediction TRP. This may comprise a quality assessment module QAM (which may also be based on a ResNet) which is configured to detect tiles and/or entire whole slide images WSI, F-WSI which are not suited for feature vector HP-VF extraction (e.g., because they do not comprise tissue or because the staining is incomplete). The number of rejected tiles (or, in general, the outcome of the quality assessment module QAM) may, for instance, be used to calculate the certainty measure CM by the certainty calculation module CCM.

In addition, the prediction function PF may comprise a certainty calculation module CCM. Specifically, the certainty calculation module CCM may be based on a Bayesian network with Monte Carlo Dropout in the individual modules of the prediction function. Taking the histopathology image analysis module HP-IAM as an example, whole slide images WSI, F-WSI may be input to a convolutional encoder-decoder. The convolutional encoder-decoder comprises a plurality of convolutional layers, batch normalisation layers and rectified linear activation function layers. These layers form different stages, also named convolutional blocks, with different resolutions of features. A new stage with a lower resolution may be generated using a pooling layer and a new stage with a higher resolution may be generated using an upsampling layer. Further, some dropout layers are each inserted between a pooling layer and following convolutional layers or between a pooling layer and an upsampling layer or between a convolutional layer and an upsampling layer. The dropout layers add a dropout after each convolutional block. These dropouts are used to determine a sample variance as an uncertainty metric. In a similar way, also the other data processing modules LLM, RA-IAM and the prediction module PM may be supplemented with dropout-layers.

In the end, a normalisation is executed for the dropouts by a Softmax-layer. Then, a stochastic dropout is performed to get stochastic dropout samples. A mean value of the stochastic dropout samples is used for determining a prediction result and a variance of the stochastic dropout samples is used for determining an uncertainty model including uncertain data so as to obtain a certainty measure CM for individual data items processed.

In other words, a Monte Carlo Dropout scheme may be implemented in which the certainty calculation module CCM is integrated in the modules of the prediction function PF in the form of the dropout layers, the Softmax-layer and the stochastic dropout. With that, dropout regularization is leveraged to produce more reliable predictions and estimate prediction certainty measures CM.

However, there are also other implementations possible, in which the certainty calculation module CCM is more separated from the actual prediction function PF, e.g., in the form of an independent module processing the output of the prediction function and/or the raw input data of the prediction function so as to estimate certainty measures CM. For instance, such independent module can take the form of a novelty detection algorithm as herein described.

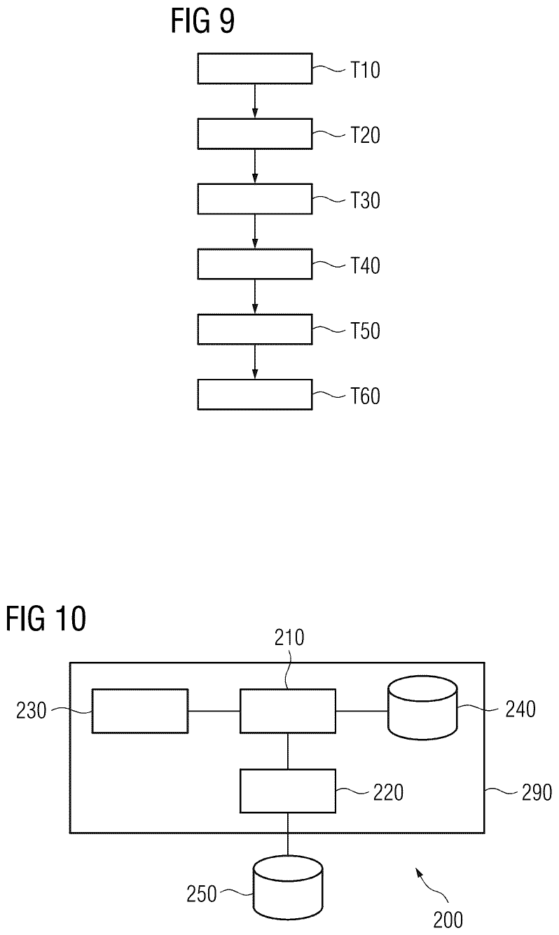

FIG. 9 depicts a method for providing a prediction function PF to predict a treatment response prediction TRP based on input data. Thereby, the input data comprises at least one whole slide image WSI and, optionally, supplementary information SI. The method comprises several steps. The order of the steps does not necessarily correspond to the numbering of the steps but may also vary between different embodiments of the present invention.

A first step T10 is directed to provide a plurality of training data sets. The training data sets respectively comprise a training input data set and a corresponding verified output data set. The training input data sets are preferably of the same type as the input data to be processed by the deployed and readily trained prediction function PF. Accordingly, the training input data sets each likewise comprise at least a whole slide image WSI. Further, the training input data sets may comprise supplementary information SI as described before. The verified output data sets comprise an indication of the treatment option(s) followed and the verified treatment response(s). Specifically, the training data sets may comprise data sets of a plurality of patients (>1000) suffering from a particular cancerous disease. The data set of each patient may respectively comprise at least one WSI and an information about the treatment option followed and the respective treatment response. The treatment response may be a long-term survival rate, a prognosis, a disease progression and so forth. The training data sets may be clustered according to the treatment options respectively followed. For each treatment option, the prediction function PF may then learn to predict the treatment response based on the respective whole slide image WSI and, optionally, the supplementary information SI.

Next, at step T20, a training data set is provided to the (not readily trained) prediction function PF.

Based on the training input data of the training data set, the prediction function PF will determine an intermediate treatment response prediction according to the learned task in step T30.

The performance of the prediction function PF (i.e., the quality of the intermediate treatment response prediction) is evaluated in subsequent step T40 based on a comparison of the verified treatment response and the intermediate treatment response prediction.

The comparison is used as a loss function to adjust weights of the prediction function PF at step T50.

At step T60 the steps of obtaining an intermediate treatment response prediction (step T30) and comparing the result to the verified treatment response (step T40) are repeated with paired sets of training input data sets and output data sets until the prediction function PF is able to generate results that are acceptable (i.e., until a local minimum of the loss function is reached). Once all pairs have been used, pairs are randomly shuffled for the next pass.

FIG. 10 illustrates an embodiment of a system 200 for providing (training) a prediction function PF. The system comprises a processor 210, an interface 220, a memory 230, a storage 240, and a database 250. Processor 210, interface 220, memory 230 and storage 240 may be embodied by a computer 290. Processor 210 controls the overall operation of the computer 200 by executing computer program instructions which define such operation. The computer program instructions may be stored in memory 230 or in storage 240 and loaded into memory 230 when execution of the computer program instructions is desired. Storage 240 may be a local storage as a component of the system 200, or a remote storage accessible over a network, such as a component of a server or cloud system. The method steps illustrated in FIG. 8 may be defined by the computer program instructions stored in memory 230 and/or storage 240, and controlled by processor 210 executing the computer program instructions.

Database 250 is a storage device such a cloud or local storage serving as an archive for the training data sets as introduced above. Database 250 may be connected to computer 290 for receipt of one or more medical images. It is also possible to implement database 250 and computer 290 as a single device. It is further possible that database 250 and computer 290 communicate wirelessly or with wired connection through a network. Interface 220 is configured to interact with database 250.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuity such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Wherever meaningful, individual embodiments or their individual aspects and features can be combined or exchanged with one another without limiting or widening the scope of the present invention. Advantages which are described with respect to one embodiment of the present invention are, wherever applicable, also advantageous to other embodiments of the present invention. Independent of the grammatical term usage, individuals with male, female or other gender identities are included within the term.

Although the present invention has been shown and described with respect to certain example embodiments, equivalents and modifications will occur to others skilled in the art upon the reading and understanding of the specification. The present invention includes all such equivalents and modifications and is limited only by the scope of the appended claims.

What is claimed is:

1. A computer-implemented method for providing a treatment response prediction for a patient suffering from a cancerous disease, the computer-implemented method comprising:
obtaining a whole slide image of the patient, the whole slide image showing a tissue sample relating to the cancerous disease;
providing a prediction function configured to derive, from whole slide images, a treatment response prediction for one or more treatment options;
applying the prediction function to the whole slide image to generate the treatment response prediction; and
providing the treatment response prediction.

2. The computer-implemented method according to claim 1, wherein the one or more treatment options comprise at least one of:
a radiotherapy treatment,
an immunotherapy treatment,
a chemotherapy treatment, or
a treatment by surgical intervention.

3. The computer-implemented method according to claim 1, wherein the treatment response prediction comprises at least one of:
a predicted susceptibility of the cancerous disease to the one or more treatment options,
a probability for a reoccurrence of the cancerous disease based on the one or more treatment options, or
a predicted survival rate of the patient with the one or more treatment options.

4. The computer-implemented method according to claim 1, further comprising:
providing a certainty calculation module configured to output a certainty measure for a corresponding treatment response prediction, the certainty measure measuring a confidence of the corresponding treatment response prediction;
applying the certainty calculation module to obtain a certainty measure for the treatment response prediction; and
providing the certainty measure for the treatment response prediction.

5. The computer-implemented method according to claim 4, wherein the providing the certainty measure comprises:
outputting the certainty measure together with the treatment response prediction to a user via a user interface.

6. The computer-implemented method according to claim 4, further comprising:
determining, based on the certainty measure, whether or not the treatment response prediction is conclusive; and
in response to the treatment response prediction being inconclusive
determining a piece of information relating to the patient that is different than the whole slide image and which is suited for rendering the treatment response prediction conclusive.

7. The computer-implemented method according to claim 6, further comprising:
accessing a healthcare information system including healthcare data of the patient;
retrieving the piece of information from the healthcare information system;
processing the piece of information to provide an updated treatment response prediction and an updated certainty measure; and
providing the updated treatment response prediction and the updated certainty measure.

8. The computer-implemented method according to claim 6, further comprising:
accessing a healthcare information system comprising healthcare data of the patient;
determining whether the piece of information is available in the healthcare information system; and
generating, via a user interface, a corresponding notification to a user in response to the piece of information not being available.

9. The computer-implemented method according to claim 6, wherein
the piece of information relates to a medical examination of the patient, and
the method further includes
generating an instruction for performing the medical examination, and
providing the instruction.

10. The computer-implemented method according to claim 1, further comprising:
obtaining, from a healthcare information system, supplementary information associated with the patient, wherein
the prediction function is further configured to derive the treatment response prediction additionally based on the supplementary information, and
the applying includes additionally applying the prediction function to the supplementary information.

11. The computer-implemented method according to claim 10, further comprising:
providing a data extraction module configured to search an electronic medical record of the patient for the supplementary information, the data extraction module including a large language model, and wherein
the obtaining of the supplementary information includes
accessing the electronic medical record of the patient in the healthcare information system, and
applying the data extraction module to the electronic medical record to obtain the supplementary information.

12. The computer-implemented method according to claim 10, wherein the supplementary information includes radiology image data depicting a manifestation of the cancerous disease in a body of the patient.

13. The computer-implemented method according to claim 12, further comprising:
providing an image analysis module configured to extract a radiological observable from radiology image data; and
applying the image analysis module on the radiology image data to obtain the radiological observable, wherein
the prediction function is further configured to derive the treatment response prediction additionally based on the radiological observable, and
the applying includes additionally applying the prediction function to the radiological observable.

14. The computer-implemented method according to claim 13, wherein the radiological observable comprises at least one of:
a tumor burden of the patient in the radiological image data,
a visual characteristic of a lesion depicted in the radiological image data, or
a temporal evolution of a lesion depicted in the radiological image data.

15. The computer-implemented method according to claim 1, wherein the one or more treatment options comprise at least two different radiotherapy treatment options, the at least two different radiotherapy treatment options differing in at least one of:
a dose distribution,
a dose threshold limit,
a dose rate,
a fractionation,
a usage of proton/photon or electron radiation, or
a usage of a co-planar or non-co-planar beam.

16. The computer-implemented method according to claim 1, wherein the one or more treatment options includes a radiotherapy treatment with a dose rate greater than 40 Gy/sec.

17. A system for providing a treatment response prediction for a patient suffering from a cancerous disease, the system comprising:
an interface unit configured to obtain a whole slide image of the patient, the whole slide image showing a tissue sample relating to the cancerous disease, and
a computing unit configured to
host a prediction function configured to derive, from whole slide images, a treatment response prediction for one or more treatment options,
apply the prediction function to the whole slide image to generate the treatment response prediction, and
provide the treatment response prediction via the interface unit.

18. A non-transitory computer program product comprising program elements that induce a computing unit of a system to perform the method of claim 1, when the program elements are loaded into a memory of the computing unit.

19. A non-transitory computer-readable storage medium storing computer-executable instructions that, when executed by at least one processor of a system, cause the system to perform the method according to claim 1.

20. The computer-implemented method according to claim 5, further comprising:
determining, based on the certainty measure, whether or not the treatment response prediction is conclusive; and
in response to the treatment response prediction being inconclusive
determining a piece of information relating to the patient that is different than the whole slide image and which is suited for rendering the treatment response prediction conclusive.

21. The computer-implemented method according to claim 6, further comprising:
accessing a healthcare information system including healthcare data of the patient;
retrieving the piece of information from the healthcare information system;
processing the piece of information to provide an updated treatment response prediction; and
providing the updated treatment response prediction.
